(19) United States
(12) Patent Application Publication
(10) Pub. No.: US 2010/0028975 A1
(43) Pub. Date: Feb. 4, 2010

(54) TRAITS IN RECOMBINANT XYLOSE-GROWING SACCHAROMYCES CEREVISIAE STRAINS USING GENOME-WIDE TRANSCRIPTION ANALYSIS

(76) Inventor: Marie-Franciose Gorwa-Grauslund, Klagshamn (SE)

Correspondence Address: GAUTHIER & CONNORS, LLP, 225 FRANKLIN STREET, SUITE 2300, BOSTON, MA, 02110, US

(21) Appl. No.: 12/548,786

(22) Filed: Aug. 27, 2009

Related U.S. Application Data
(63) Continuation of application No. 11/522,888, filed on Sep. 18, 2006, now abandoned.
Application No. 11/522,888 is a continuation of application No. PCT/SE2005/000445, filed on Mar. 24, 2005.

(30) Foreign Application Priority Data
Mar. 26, 2004 (SE) 0400815-7

(51) Int. Cl. C12N 1/19

(52) U.S. Cl. 435/254.21

(57) ABSTRACT

New xylose-utilizing Saccharomyces cerevisiae strain which can ferment xylose to ethanol. It expresses the gene for A) xylose reducatse (XR) and Xylitol dehydrogenase (XDH) or B) xylose isomerase (XI) and has a) increased transporting capacity with regard to xylose, b) increased conversion capacity of xylulose to xylulose-5P, c) increased activity of the oxidative pentose phosphate pathway, and/or d) increased activity of the non-oxidative pentose phosphate pathway.

### TECHNICAL FIELD

The present invention relates to novel recombinant Saccharomyces cerevisiae strains utilizing pentoses, such as xylose, for the production of ethanol.

### BACKGROUND OF THE INVENTION

Metabolic engineering has been a valuable tool for enhancing ethanol yield and productivity from xylose in recombinant Saccharomyces cerevisiae (Hahn-Hägerdal et al., 2001). However, to date, strains constructed by genetic engineering of laboratory strains do not display high xylose growth rate and xylose consumption rate, two properties that would enhance the economic feasibility of a biofuel ethanol process. By approaching this problem starting with recombinant yeast strains and exposing them to random mutagenesis (Wahlbom et al., 2003a), adaptation (Sonderegger and Sauer, 2003) and breeding (Spencer-Martins, 2003), a number of xylose growing strains have been generated. TMB3400 has been selected for xylose growth and fermentation after chemical mutagenesis of TMB3399 (Wahlbom et al., 2003); C1 and C5 have been evolved from TMB3001 (Eliasson et al., 2000b) by adaptation to anaerobic conditions on xylose in continuous culture and EMS mutagenesis (Sonderegger and Sauer, 2003), and BH42 has been obtained from TMB3001 and other xylose-utilizing S. cerevisiae strains by breeding (Spencer-Martins, 2003). F12 has been obtained by transformation of the industrial strain F with the xylose pathway genes (Sonderegger et al., 2004b). These strains display enhanced aerobic xylose growth rates but the gene modification(s) that are responsible for this property are not known.

Genome-wide transcription analysis is a valuable tool to identify changes in gene expression level. It has been used in S. cerevisiae to identify genes whose expression level is changed by different cultivation conditions, such as the oxygenation level (ter Linde et al., 1999), cobalt stress (Stadler and Schweyen, 2002) or sugar-induced osmotic stress (Erasmus et al., 2003). The identification of genes whose expression is controlled by another gene is also possible, as shown for GAL4 (Ren et al., 2000; Bro et al., 2004) that is involved in the regulation of galactose metabolism, and STE12 (Ren et al., 2000) involved in mating metabolism. Xylose-utilizing S. cerevisiae strains have been analyzed by genome-wide transcription analysis (Sedlak et al., 2003; Sonderegger et al., 2004a; Wahlbom et al., 2003b). Enhanced mRNA levels were found in the pentose phosphate pathway, the xylose pathway and in sugar transport for the mutant TMB3400 compared to its parental strain TMB3399 (Wahlbom et al., 2003b). The anaerobic xylose-growing C1 strain displayed significantly changed expression levels in the xylose pathway, the pentose phosphate pathway and the glycerol pathway (Sonderegger et al., 2004a). Furthermore, C1 displayed increased transcript levels for genes increasing cytosolic NADPH formation and NADH consumption. In addition, mRNA levels for genes in the glycolytic and alcoholic pathways in a xylose-utilizing S. cerevisiae strain have been analyzed (Sedlak et al., 2003).

### SUMMARY OF THE PRESENT INVENTION

In contrast to previous studies in which a single strain was compared to its parental strain, the present investigation aimed at combining genome-wide transcription analyses for several strains in a single study with the objective to identify common specific traits. S. cerevisiae strains C1, C5 (Sonderegger and Sauer, 2003), TMB3001 (Eliasson et al., 2000b), TMB3400, TMB3399 (Wahlbom et al., 2003a), BH42 (Spencer-Martins, 2003) and F12 (Sonderegger et al., 2004b) were used. Aerobic xylose consumption and maximum specific growth rate on xylose were measured. Open reading frames (ORFs) with changed expression levels in the xylose-growing strains were selected based on SLR- and p-values obtained from the comparison analysis in MicroArray Suite 5.0 (MAS 5.0).

In particular the present invention relates to a new xylose-utilizing Saccharomyces cerevisiae strain by expression of xylose reductase (XR-XDH) or xylose isomerase (XI) genes fermenting xylose to ethanol better than a control strain having
a) increased transporting capacity with regard to xylose,
b) increased conversion capacity of xylulose to xylulose-5P
c) increased activity of the oxidative pentose phosphate pathway, and/or
d) increased activity of the non-oxidative pentose phosphate pathway,

In a preferred embodiment of the strain the gene GAL2 is up-regulated to provide for an increased level of the Gal2p permease.

In a preferred embodiment of the strain the gene XKS1 is up-regulated.

In a preferred embodiment of the strain the genes SOL1, SOL2, SOL3, SOL4, ZWF1 and/or GND1 are up-regulated to provide for an increased level of glucose-6-phosphatase dehydrogenase, and phosphogluconate dehydrogenase.

In a preferred embodiment of the strain the gene TAL1 is upregulated to provide for an increased level of transaldolase, the gene TKL1 to provide for an increased level of transketolase, the gene RPE1 to provide for an increased level of D-ribulose-5-phosphate-3-epimerase, and/or the gene RKI1 to provide for an increased level of D-ribose-5-phosphate ketol-isomerase.

In a preferred embodiment of the strain the gene YEL041W to provide for an increased level of NAD(H)⁺ kinase.

In a preferred embodiment of the strain the genes GAL1, GAL7 and GAL10 are up-regulated.

In a preferred embodiment of the strain the gene PUT4 is upregulated.

In a preferred embodiment of the strain the gene YLR152c is up-regulated.

In a preferred embodiment of the strain the gene YOR202W is up-regulated.

In a preferred embodiment of the strain two or more properties of above are combined.

### DETAILED DESCRIPTION OF THE PRESENT INVENTION

### Materials and Methods

### Strains.

Strains used in the present investigation are summarized in Table 1.

### Continuous Cultivation of F12, BH42 and C5.

Aerobic continuous cultures were conducted in a Biostat® bioreactor (B. Braun Biotech International, Melsungen, Germany) at a dilution rate of 0.1 h⁻¹. A total volume of 1200 ml defined mineral medium (Verduyn et al., 1992) with double concentration of all components except KH₂PO₄ was used. Antifoam (Dow Corning® Antifoam RD Emulsion, BDH Laboratory Supplies, Poole, England) was added at a concentration of 0.5 ml l⁻¹. The carbon source consisted of 10 g/l glucose or a mixture of 10 g l⁻¹ glucose and 10 g l⁻¹ xylose. The temperature was 30° C., the pH 5.5 (controlled by 3M KOH) and aerobic conditions were ensured by sparging with 1 l min⁻¹ air and a stirring speed of 1000 rpm. Dissolved oxygen was kept above 75% at all times. Steady state was assumed after at least 6 fermentor volumes had passed. TMB3001, C1, TMB3399 and TMB3400 have previously been cultivated in continuous mode using the same medium (Verduyn et al., 1992) at the dilution rates and substrate concentrations presented in Table 2 (Sonderegger et al., 2004a; Wahlbom et al., 2003b). C5 was cultivated in the same manner as C1 and with 20 g l⁻¹ xylose.

### Growth Fates.

Overnight-cultures with 10 g l⁻¹ glucose and 10 g l⁻¹ xylose in defined mineral medium (Verduyn et al., 1992) were used to inoculate the same medium containing 20 g l⁻¹ xylose in a baffled shake-flasks filled to ⅕ of the total volume to an OD620 of 0.2. Maximum specific growth rates were measured for all strains at 30° C. and a stirring speed of 140 rpm.

### Sampling.

Substrate consumption and product formation was measured by HPLC as previously described (Jeppsson et al., 2002). Outgoing gas composition was monitored with a Carbon Dioxide and Oxygen Monitor Type 1308 (Brüel&Kjaer, Copenhagen, Denmark) and biomass was measured after filtering 1 volume of sample and 3 volumes of water through pre-weighed 0.45 μm filters, which were then dried in a microwave oven at 350 W for 8 min.

### Microarray Experiments.

Cells for RNA isolation were harvested by centrifugation at 5000 g for 5 min at 4° C. The cells were washed with ice-cold AE-buffer, frozen in liquid nitrogen and stored at −80° C. until processed further. RNA was isolated using the hot phenol method (Schmitt et al., 1990). Purification of mRNA, cDNA synthesis, in vitro transcription, and fragmentation were performed as described (Affymetrix). Hybridization, washing, staining and scanning of microarray-chips (Yeast Genome S98 Arrays) was made with a Hybridization Oven 320, a Fluidics Station 400 and a GeneArray Scanner (Affymetrix), respectively.

### Data Quality.

Quality of the RNA expression data was assessed by calculating the average coefficient of variation (the average of the standard deviation divided by the mean) for the two signals obtained for each yeast ORF. Then, the means of the coefficients of variation for all yeast ORFs were calculated, resulting in average coefficients of variation of 0.12-0.34 for the different strains (Table 2). These values are in the same range as the previously obtained average intra-laboratory coefficient of variation of 0.23 for 86% of the most highly expressed yeast genes in glucose-limited chemostat cultures (Piper et al., 2002).

### Comparison Analysis.

Data was processed with Affymetrix Microarray Suite (MAS 5.0) and sorted in Microsoft Excel. Default parameters were used for expression analysis settings in MAS 5.0. A normalization value of 1 (user defined) and a scaling factor of 100 (all probes set) was used. In MAS 5.0, single array analysis gives a detection call (Present/Absent) and a signal value which is a relative measure of abundance of the transcript. The values reported in the present investigation are average signals of gene expression on duplicate samples. Genes with changed expression levels were selected based on Signal Log Ratio (SLR) or p-values obtained in a comparison analysis in MAS 5.0. For each set of two strains (A and B) and one condition, 4 comparisons were made including duplicate samples of each strain and condition (A1 vs B1, A2 vs B1, A1 vs B2 and A2 vs B2) (Affymetrix, 2003).

The SLR-value, calculated by comparing each probe pair on the experiment array to the corresponding probe pair on the base-line array, indicates magnitude and direction of change of a transcript (Affymetrix, 2003). It is based on the logarithm with base two, and therefore the fold change is 2^{SLR} at SLR higher or equal to 0 and it is −2^{-SLR} at SLR<0. The p-value is the probability that an observation occurs by chance under the null hypothesis (Affymetrix, 2002), and the change p-value in MAS 5.0 indicates the probability for change and the direction of it when the transcripts on two arrays are compared. The change call (Increase, Decrease, No change) is based on the p-value.

Double criteria, including both SLR and p-value, were used in the strain comparisons in Tables 4 and 5. When for example an absolute SLR value of 1.0 was used as cut-off value, only ORFs where SLR was either higher or equal to 1.0, or lower or equal to −1.0 in all comparisons (4 per strain and condition) were kept. When the detection call was “Absent” for at least one signal in the pair with the higher signals or the change call was not I (=increased) or D (=decreased) for all comparisons, the gene expression was not considered changed even though it had been selected for a certain absolute SLR-value. In Table 3 and Tables 6-9, however, only the change call was used for selection of genes with changed expression levels, in order to select genes based on changed expression levels but not necessarily high SLR-values.

### Results

### Aerobic Xylose Consumption and Maximum Specific Growth Rate.

The maximum aerobic specific growth rate on xylose was determined under the same conditions for all improved xylose-growing strains (C1, C5, BH42, TMB3400, F12) and parental strains (TMB3001, TMB3399) (Table 1) and was then compared with the xylose consumption in aerobic continuous culture (Table 2). Higher xylose growth rate correlated with higher xylose consumption. TMB3399, F12, TMB3400 and BH42 consumed 5.4, 6.4, 7.1 and 7.8 g l⁻¹ xylose (Table 2) in continuous culture with 10 g/l glucose and 10 g l⁻¹ xylose at dilution rate 0.1 h⁻¹, while having maximum specific growth rates on xylose of 0.09, 0.13, 0.17 and 0.20 h⁻¹, respectively (Table 1). TMB3001 and C1 consumed 4.2 and 9.6 g l⁻¹ xylose (Table 2) in continuous culture with 10 g l⁻¹ glucose and 10 g l⁻¹ xylose at dilution rate 0.05 h⁻¹, and had maximum specific growth rates of 0.09 and 0.21 h⁻¹ on xylose (Table 1). C5, which was only cultivated on xylose in continuous cultivation, had a maximum specific xylose growth rate of 0.14 h⁻¹.

TABLE 1
Strains used in this study (with their original reference into parentheses) and their
maximum specific aerobic growth rates on xylose (μmax). Results for μmax
are mean values and deviation from mean from duplicate experiments. After 20 h,
the maximum specific growth rate rapidly decreased for all strains.

| | | μmax xylose |
|---|---|---|
| Strain | Relevant genotype | (h⁻¹) |
| TMB3399 (Wahlbom et al., 2003a) | USM21 HIS3::YIpXR/XDH/XK | 0.09 ± 0.1 |
| | (Industrial, polyploid strain) | |
| TMB3400 (Wahlbom et al., 2003a) | Isolated after mutagenesis and selection for xylose growth and | 0.17 ± 0.1 |
| | fermentation of TMB3399 | |
| BH42 (Spencer-Martins, 2003) | Strain with improved xylose growth resulting from breeding | 0.20 ± 0.1 |
| F12 (Sonderegger et al., 2004b) | S. cerevisiae F HIS3::YIploxZEO, overexpressing XR, XDH, and | 0.13 ± 0.1 |
| | XK (Industrial, polyploid strain) | |
| TMB3001 (Eliasson et al., 2000b) | CEN.PK 113-7A (MATα his3-Δ1 | 0.09 ± 0.1 |
| | MAL2-8c SUC2) his3::YIp XR/XDH/XK | |
| C1 (Sonderegger and Sauer, 2003) | Clone isolated from TMBEP (Evolved population of TMB3001) | 0.21 ± 0.1 |
| C5 (Sonderegger and Sauer, 2003) | Clone isolated from TMBEP (Evolved population of TMB3001) | 0.14 ± 0.1 |

TABLE 2
Consumed substrates and formed products (g l⁻¹ glucose, xylose, biomass and CO₂), C-balances, dilution rates (h⁻¹) and average
coefficients of variation (average of standard deviations/means) for mRNA level signals in aerobic continuous culture in defined medium with
10 g l⁻¹ glucose, 10 g l⁻¹ glucose and 10 g l⁻¹ xylose or 20 g l⁻¹ xylose as carbon source.

| | Consumed | Consumed | | | | | | |
|---|---|---|---|---|---|---|---|---|
| | Glucose | Xylose | | | | Dilution | Average | Reference |
| | (glucose left) | (xylose left) | Biomass | CO₂ | C- | rate | coefficient | cultivation |
| Strain | g l⁻¹ | g l⁻¹ | g l⁻¹ | g l⁻¹ | balance % | h⁻¹ | of variation | data |
| TMB3399 | 10.8 ± 0.2 (ND) | — | 5.1 ± 0.2 | 7.29 ± 0.36 | 96 | 0.1 | 0.23 | (Wahlbom et al., 2003b) |
| TMB3399 | 10.2 ± 0.1 (0.09) | 5.4 ± 0.3 (4.6) | 7.4 ± 0.1 | 11.10 ± 0.07 | 101 | 0.1 | 0.22 | (Wahlbom et al., 2003b) |
| TMB3400 | 10.8 ± 0.2 (ND) | — | 4.9 ± 0.2 | 6.66 ± 0.05 | 94 | 0.1 | 0.21 | (Wahlbom et al., 2003b) |
| TMB3400 | 10.2 ± 0.1 (ND) | 7.1 ± 0.6 (2.9) | 8.1 ± 0.4 | 12.56 ± 0.24 | 103 | 0.1 | 0.21 | (Wahlbom et al., 2003b) |
| TMB3400 | — | 12.3 ± 0.2 (8.5) | 5.4 ± 0.1 | 8.80 ± 0.05 | 94 | 0.1 | 0.20 | (Wahlbom et al., 2003b) |
| BH42 | 11.0 ± 0.0 (0.05) | — | 6.8 ± 0.2 | 7.14 ± 0.07 | 112 | 0.1 | 0.16 | This work |
| BH42 | 10.8 ± 0.1 (0.06) | 7.8 ± 0.3 (2.7) | 11.5 ± 0.5 | 12.88 ± 1.84 | 113 | 0.1 | 0.20 | This work |
| F12 | 11.0 ± 0.2 (0.06) | — | 5.7 ± 0.2 | 7.98 ± 0.68 | 103 | 0.1 | 0.12 | This work |
| F12 | 10.6 ± 0.5 (0.06) | 6.4 ± 0.3 (3.6) | 9.4 ± 0.7 | 13.16 ± 1.13 | 116 | 0.1 | 0.15 | This work |
| TMB3001 | 9.6 ± 0.1 (ND) | 4.2 ± 0.7 (5.5) | 6.5 ± 0.3 | 11.7 ± 0.0 | 114 | 0.05 | 0.16* | (Sonderegger et al., |
| | | | | | | | | 2004a) |
| C1 | 9.6 ± 0.1 (ND) | 9.6 ± 0.1 (0.1) | 10.0 ± 0.2 | 14.0 ± 0.0 | 115 | 0.05 | 0.13* | (Sonderegger et al., |
| | | | | | | | | 2004a) |
| C1 | — | 20.1 ± 0.2 (0.8) | 8.7 ± 0.0 | 13.9 ± 0.0 | 100 | 0.05 | 0.34* | (Sonderegger et al., |
| | | | | | | | | 2004a) |
| C5 | — | 19.9 ± 1.8 (0.7) | 7.8 ± 0.8 | 14.0 ± 0.0 | 95 | 0.05 | 0.23 | This work |

ND: Not detected,
*These numbers were also reported by Sonderegger et al. (2004a).

Gene expression levels of strains C1, C5, BH42, F12 and TMB3400, which have maximum specific xylose growth rates of 0.13-0.21 h⁻¹, were compared with gene expression levels of TMB3001 or TMB3399 which grow at 0.09 h⁻¹.

Changes in transport and central metabolism. The xylose transport step and the central metabolism, which are involved in the conversion of xylose to ethanol, are likely to be affected when xylose growth is enhanced. For example, the non-oxidative pentose phosphate pathway has previously been shown to limit xylulose fermentation rate in a recombinant XR/XDH/XK overproducing S. cerevisiae strain (Johansson and Hahn-Hägerdal, 2002). A comparison was therefore performed using all the strains in order to search for specific or general traits within these steps. The comparison was performed on aerobically glucose-xylose grown strains, except for C5 which had been cultivated on xylose only. C1 and BH42 were compared to TMB3001, whereas TMB3400 was compared to TMB3399. C5 (xylose grown) was compared to TMB3001 (glucose-xylose grown). Only genes with solely change call I (increase) or D (decrease) in at least one comparison are shown in Table 3. F12, which does not have a control strain, was not included when selecting for changed gene levels but its signals were included in Tables 3a and 3b.

Decreased mRNA expression levels of HXT2, HXT3, HXT4, HXT5, and MAL11, encoding hexose transporters, were observed in C1 and C5 (Table 3a). MAL11 was also down-regulated in BH42. GAL2, encoding galactose permease, was strongly up-regulated (60-210 fold on signal) in C1, C5 and BH42, and had a high expression in F12 compared to TMB3001 and TMB3399. TMB3400 did not display enhanced expression levels for any transporters compared to TMB3399 when grown on a glucose/xylose mixture. However, when the expression levels were compared for TMB3400 on xylose and TMB3399 on glucose, GAL2 was enhanced about 70 times.

The expression of xylose pathway genes can only be partly investigated, since the integrated P. stipitis XYL1 and XYL2 genes were not included on the microarrays. The signal for the GRE3 gene, encoding an S. cerevisiae protein capable of xylose reduction (Kuhn et al., 1995; Träff et al., 2002), was higher for F12 than for TMB3001 (2.5 fold on signal). S. cerevisiae XYL2, encoding xylitol dehydrogenase, was up-regulated in BH42 only, and XKS1, encoding xylulokinase, had enhanced expression level in C1 and C5 (Table 3a) and in xylose-utilising TMB3400 (data not shown).

Both the oxidative (ZWF1, GND1, SOL2, SOL3) and non-oxidative (TAL1, TKL1) pentose phosphate pathway (PPP) genes were up-regulated in C1, C5 and BH42 compared to TMB3001 (Table 3a). In TMB3400 only the oxidative PPP (GND1, SOL3) was up-regulated compared to TMB3399. However, in TMB3399 the expression level of the non-oxidative PPP genes was already high, in the same range as in the C1, C5 and BH42 strains. F12 also had high expression levels for both the non-oxidative and oxidative PPP. PPP genes were up-regulated in BH42 and TMB3400 when grown on a mixture of glucose and xylose, and were also up-regulated when glucose was used as the sole carbon source (data not shown), indicating that the up-regulated PPP is constitutive and not a result of xylose induction.

The glycolytic genes PYK2, encoding pyruvate kinase, and YDR516C, encoding a protein similar to glucokinase, were up-regulated in C1, C5 and BH42 (Table 3b). A number of other glycolytic genes displayed enhanced expression levels in one or two of the xylose growing strains. The glycerol pathway was enhanced in C1, C5 and BH42: GPD1 was up-regulated in BH42, whereas GPD2 and RHR2 were up-regulated in C1 and C5.

Up-regulations were also found for genes encoding pyruvate decarboxylase and alcohol dehydrogenase activities (Table 3b). C1 and C5 displayed enhanced levels of ADH4, ADH5 and PDC6, and also the level of ADH6 was enhanced in C1. BH42 showed increased levels of ADH5, ADH6, ADH7 and PDC5. Changed expression levels were also observed for genes encoding aldehyde dehydrogenases.

TABLE 3a
Signals for changed gene expression levels (=change call solely I or D for at least one strain compared with its reference strain) in
transport, xylose pathway and pentose phosphate pathway (PPP). Signals for up-regulated genes are written in bold, and signals for down-
regulated genes are written in italic. Strains were cultivated on glucose (g), glucose and xylose (gx) or xylose alone (x).

| | | TMB300 | | | | | | |
|---|---|---|---|---|---|---|---|---|
| | | 1 gx | | | | | TMB3399 | |
| | | Ref. | | | | | gx | TMB3400 |
| ORF | Affymetrix annotation | strain | C1 gx | C5 x | BH42 gx | F12 gx | Ref. strain | gx |
| Transport | | | | | | | | |
| YMR011W | HXT2, Hexose transporter | 1147 ± 70 | 406 ± 2 | 514 ± 105 | 1017 ± 336 | 1121 ± 74 | 2037 ± 37 | 1965 ± 634 |
| | (high affinity | | | | | | | |
| | glucose transporter) | | | | | | | |
| YDR345C | HXT3, Hexose transporter | 164 ± 23 | 91 ± 10 | 84 ± 25 | 150 ± 1 | 102 ± 1 | 114 ± 18 | 158 ± 61 |
| | (low/high affinity | | | | | | | |
| | glucose transporter) | | | | | | | |
| YHR092C | HXT4, Hexose transporter | 168 ± 30 | 50 ± 10 | 32 ± 4 | 150 ± 47 | 98 ± 5 | 100 ± 76 | 35 ± 1 |
| | (high-affinity | | | | | | | |
| | glucose transporter) | | | | | | | |
| YHR096C | HXT5, Hexose transporter | 656 ± 6 | 267 ± 15 | 249 ± 20 | 761 ± 113 | 1464 ± 168 | 506 ± 142 | 655 ± 64 |
| YFL011W | HXT10, Hexose transporter | 7 ± 1 | 2 ± 1 | 3 ± 1 | 3 ± 1 | 3 ± 1 | 2 ± 1 | 4 ± 1 |
| YJR158W | HXT16, Hexose transporter | 11 ± 1 | 91 ± 4 | 272 ± 66 | 18 ± 7 | 9 ± 1 | 18 ± 1 | 17 ± 1 |
| YGR289C | MAL11, Hexose transporter | 421 ± 113 | 85 ± 4 | 56 ± 8 | 158 ± 65 | 71 ± 21 | 23 ± 1 | 25 ± 2 |
| | (maltose permease) | | | | | | | |
| YDR536W | STL1, Sugar transporter-like | 143 ± 59 | 19 ± 4 | 50 ± 10 | 93 ± 4 | 280 ± 44 | 64 ± 8 | 97 ± 27 |
| | protein | | | | | | | |
| YBR241C | Probable sugar transport protein | 113 ± 12 | 107 ± 11 | 116 ± 9 | 388 ± 11 | 709 ± 27 | 225 ± 23 | 216 ± 19 |
| YDL247W | MPH2, Strong similarity to | 14 ± 1 | 7 ± 1 | 5 ± 1 | 6 ± 4 | 4 ± 1 | 3 ± 2 | 5 ± 1 |
| | sugar transport proteins | | | | | | | |
| YLR081W | GAL2, Galactose permease | 8 ± 1 | 1633 ± 34 | 1651 ± 35 | 557 ± 55 | 26 ± 4 | 9 ± 1 | 6 ± 4 |
| Xylose | | | | | | | | |
| YLR070C | XYL2, Strong similarity to sugar | 56 ± 2 | 77 ± 12 | 45 ± 21 | 131 ± 17 | 33 ± 3 | 48 ± 6 | 38 ± 9 |
| | dehydrogenases | | | | | | | |
| YGR194C | XKS1, Xylulokinase | 552 ± 32 | 1011 ± 93 | 1126 ± 10 | 609 ± 79 | 574 ± 8 | 397 ± 85 | 643 ± 33 |
| Transport | | | | | | | | |
| YMR011W | HXT2, Hexose transporter | 1147 ± 70 | 406 ± 2 | 514 ± 105 | 1017 ± 336 | 1121 ± 74 | 2037 ± 37 | 1965 ± 634 |
| | (high affinity | | | | | | | |
| | glucose transporter) | | | | | | | |
| YDR345C | HXT3, Hexose transporter | 164 ± 23 | 91 ± 10 | 84 ± 25 | 150 ± 1 | 102 ± 1 | 114 ± 18 | 158 ± 61 |
| | (low/high affinity | | | | | | | |
| | glucose transporter) | | | | | | | |
| YHR092C | HXT4, Hexose transporter | 168 ± 30 | 50 ± 10 | 32 ± 4 | 150 ± 47 | 98 ± 5 | 100 ± 76 | 35 ± 1 |
| | (high-affinity | | | | | | | |
| | glucose transporter) | | | | | | | |
| YHR096C | HXT5, Hexose transporter | 656 ± 6 | 267 ± 15 | 249 ± 20 | 761 ± 113 | 1464 ± 168 | 506 ± 142 | 655 ± 64 |
| YFL011W | HXT10, Hexose transporter | 7 ± 1 | 2 ± 1 | 3 ± 1 | 3 ± 1 | 3 ± 1 | 2 ± 1 | 4 ± 1 |
| YJR158W | HXT16, Hexose transporter | 11 ± 1 | 91 ± 4 | 272 ± 66 | 18 ± 7 | 9 ± 1 | 18 ± 1 | 17 ± 1 |
| YGR289C | MAL11, Hexose transporter | 421 ± 113 | 85 ± 4 | 56 ± 8 | 158 ± 65 | 71 ± 21 | 23 ± 1 | 25 ± 2 |
| | (maltose permease) | | | | | | | |
| YDR536W | STL1, Sugar transporter-like | 143 ± 59 | 19 ± 4 | 50 ± 10 | 93 ± 4 | 280 ± 44 | 64 ± 8 | 97 ± 27 |
| | protein | | | | | | | |
| YBR241C | Probable sugar transport protein | 113 ± 12 | 107 ± 11 | 116 ± 9 | 388 ± 11 | 709 ± 27 | 225 ± 23 | 216 ± 19 |
| YDL247W | MPH2, Strong similarity to | 14 ± 1 | 7 ± 1 | 5 ± 1 | 6 ± 4 | 4 ± 1 | 3 ± 2 | 5 ± 1 |
| | sugar transport proteins | | | | | | | |
| YLR081W | GAL2, Galactose permease | 8 ± 1 | 1633 ± 34 | 1651 ± 35 | 557 ± 55 | 26 ± 4 | 9 ± 1 | 6 ± 4 |
| Xylose | | | | | | | | |
| YLR070C | XYL2, Strong similarity to sugar | 56 ± 2 | 77 ± 12 | 45 ± 21 | 131 ± 17 | 33 ± 3 | 48 ± 6 | 38 ± 9 |
| | dehydrogenases | | | | | | | |
| YGR194C | XKS1, Xylulokinase | 552 ± 32 | 1011 ± 93 | 1126 ± 10 | 609 ± 79 | 574 ± 8 | 397 ± 85 | 643 ± 33 |
| PPP | | | | | | | | |
| YNL241C | ZWF1, Glucose-6-phosphate | 181 ± 27 | 485 ± 12 | 526 ± 38 | 323 ± 32 | 551 ± 7 | 270 ± 27 | 284 ± 4 |
| | dehydrogenase | | | | | | | |
| YHR183W | GND1, Phosphogluconate | 831 ± 107 | 1358 ± 54 | 1729 ± 291 | 1470 ± 31 | 1311 ± 16 | 1187 ± 97 | 1775 ± 78 |
| | dehydrogenase | | | | | | | |
| | (decarboxylating) | | | | | | | |
| YNR034W | SOL1, shows similarity to | 17 ± 1 | 22 ± 3 | 14 ± 4 | 26 ± 1 | 54 ± 8 | 32 ± 2 | 28 ± 6 |
| | glucose-6-phosphate | | | | | | | |
| | dehydrogenase | | | | | | | |
| | non-catalytic domains, | | | | | | | |
| | homologous to Sol2p and Sol3p | | | | | | | |
| YCR073W-A | SOL2, Shows similarity to | 170 ± 1 | 262 ± 7 | 266 ± 38 | 294 ± 26 | 382 ± 1 | 252 ± 23 | 233 ± 15 |
| | glucose-6-phosphate | | | | | | | |
| | dehydrogenase | | | | | | | |
| | non-catalytic domains, | | | | | | | |
| | homologous to Sol1p and Sol3p | | | | | | | |
| YHR163W | SOL3, Shows similarity to | 496 ± 87 | 1068 ± 45 | 1121 ± 36 | 1072 ± 109 | 620 ± 19 | 621 ± 105 | 1274 ± 3 |
| | glucose-6-phosphate | | | | | | | |
| | dehydrogenase | | | | | | | |
| | non-catalytic domains, | | | | | | | |
| | homologous to Sol2p and Sol1p | | | | | | | |
| YGR248W | SOL4, Similar to SOL3 | 170 ± 6 | 48 ± 6 | 58 ± 12 | 201 ± 138 | 390 ± 19 | 65 ± 23 | 67 ± 12 |
| YOR095C | RKI1, Ribose-5-phosphate | 96 ± 5 | 79 ± 2 | 39 ± 2 | 89 ± 4 | 72 ± 5 | 58 ± 2 | 68 ± 13 |
| | ketol-isomerase | | | | | | | |
| YJL121C | RPE1, D-ribulose-5- | 172 ± 6 | 249 ± 2 | 162 ± 56 | 510 ± 82 | 506 ± 1 | 592 ± 97 | 552 ± 76 |
| | Phosphate 3-epimerase | | | | | | | |
| YLR354C | TAL1, Transaldolase, | 380 ± 29 | 684 ± 45 | 845 ± 11 | 745 ± 54 | 522 ± 43 | 628 ± 40 | 792 ± 38 |
| | enzyme in the pentose | | | | | | | |
| | phosphate pathway | | | | | | | |
| YGR043C | Strong similarity to transaldolase | 190 ± 28 | 63 ± 18 | 81 ± 7 | 106 ± 58 | 139 ± 25 | 75 ± 14 | 82 ± 16 |
| YBR117C | TKL2, Transketolase, | 89 ± 9 | 35 ± 7 | 34 ± 9 | 158 ± 61 | 636 ± 20 | 70 ± 22 | 46 ± 17 |
| | homologous to Tkl1p | | | | | | | |
| YPR074C | TKL1, Transketolase 1 | 422 ± 13 | 827 ± 2 | 873 ± 35 | 972 ± 110 | 1041 ± 28 | 898 ± 62 | 1069 ± 142 |

TABLE 3b
Signals for changed gene expression levels (=change call solely I or D for at least one strain compared with its reference strain) in
glycolysis, pyruvate to ethanol and acetate pathways. Signals for up-regulated genes are written in bold, and signals for down-regulated genes
are written in italic. Strains were cultivated on glucose (g), glucose and xylose (gx) or xylose alone (x).

| | | TMB300 | | | | | | |
|---|---|---|---|---|---|---|---|---|
| | | 1 gx | | | | | TMB3399 | |
| | | Ref. | | | | | gx | TMB3400 |
| ORF | Affymetrix annotation | strain | C1 gx | C5 x | BH42 gx | F12 gx | Ref. strain | gx |
| Glycolysis | | | | | | | | |
| YFR053C | HXK1, Hexokinase I (PI) | 900 ± 14 | 691 ± 61 | 1227 ± 59 | 988 ± 52 | 1405 ± 78 | 1408 ± 33 | 1146 ± 244 |
| | (also called hexokinase A) | | | | | | | |
| YGR240C | PFK1, Phosphofructokinase | 601 ± 28 | 749 ± 36 | 1045 ± 190 | 674 ± 55 | 917 ± 36 | 688 ± 68 | 578 ± 10 |
| | alpha subunit | | | | | | | |
| YLR377C | FBP1, Fructose-1,6-bisphosphatase | 113 ± 10 | 74 ± 2 | 73 ± 15 | 528 ± 42 | 341 ± 7 | 155 ± 50 | 192 ± 5 |
| YMR205C | PFK2, Phosphofructokinase | 737 ± 45 | 922 ± 29 | 933 ± 172 | 1096 ± 16 | 1379 ± 17 | 1086 ± 112 | 1004 ± 33 |
| | beta subunit | | | | | | | |
| YDL021W | GPM2, Similar to GPM1 | 77 ± 6 | 139 ± 3 | 113 ± 7 | 54 ± 4 | 79 ± 6 | 102 ± 21 | 54 ± 7 |
| | (phosphoglycerate mutase) | | | | | | | |
| YOL056W | GPM3, Phosphoglycerate mutase | 20 ± 1 | 38 ± 4 | 32 ± 2 | 27 ± 2 | 21 ± 1 | 27 ± 1 | 37 ± 6 |
| YAL038W | PYK1, Pyruvate kinase | 1396 ± 8 | 1680 ± 14 | 1729 ± 147 | 1863 ± 8 | 2079 ± 218 | 2658 ± 96 | 2292 ± 125 |
| YOR347C | PYK2, Pyruvate kinase, | 34 ± 2 | 306 ± 1 | 260 ± 29 | 111 ± 27 | 50 ± 23 | 46 ± 6 | 63 ± 11 |
| | glucose-repressed isoform | | | | | | | |
| YDR516C | Strong similarity to glucokinase | 171 ± 12 | 337 ± 1 | 328 ± 19 | 276 ± 27 | 469 ± 23 | 440 ± 21 | 435 ± 5 |
| YCL040W | GLK1, Glucokinase | 629 ± 20 | 783 ± 26 | 1017 ± 28 | 1219 ± 6 | 1729 ± 92 | 1554 ± 49 | 1034 ± 91 |
| YJL052W | TDH1, Glyceraldehyde-3- | 917 ± 39 | 1124 ± 29 | 907 ± 183 | 1321 ± 88 | 1411 ± 41 | 1818 ± 88 | 948 ± 96 |
| | phosphate dehydrogenase 1 | | | | | | | |
| YDL022W | GPD1, Glycerol-3-phosphate | 640 ± 17 | 657 ± 28 | 643 ± 44 | 796 ± 15 | 1533 ± 35 | 929 ± 205 | 932 ± 182 |
| | dehydrogenase | | | | | | | |
| YOL059W | GPD2, Glycerol-3-phosphate | 112 ± 37 | 307 ± 75 | 493 ± 31 | 97 ± 22 | 181 ± 7 | 263 ± 36 | 167 ± 14 |
| | dehydrogenase | | | | | | | |
| | (NAD⁺) | | | | | | | |
| YIL053W | RHR2, DL-glycerol-3-phosphatase | 300 ± 32 | 619 ± 2 | 620 ± 91 | 396 ± 54 | 633 ± 6 | 557 ± 6 | 287 ± 16 |
| Pyr to EtOH | | | | | | | | |
| YGL256W | ADH4, Alcohol dehydrogenase | 130 ± 15 | 343 ± 32 | 501 ± 15 | 140 ± 11 | 259 ± 8 | 560 ± 147 | 250 ± 52 |
| | isoenzyme IV | | | | | | | |
| YBR145W | ADH5, Alcohol dehydrogenase | 122 ± 5 | 311 ± 39 | 198 ± 7 | 172 ± 11 | 413 ± 78 | 620 ± 89 | 329 ± 80 |
| | isoenzyme V | | | | | | | |
| YMR318C | ADH6, Strong similarity to | 214 ± 4 | 567 ± 15 | 296 ± 61 | 725 ± 105 | 749 ± 28 | 609 ± 139 | 1158 ± 106 |
| | alcohol-dehydrogenase | | | | | | | |
| YCR105W | ADH7, Alcohol dehydrogenase | 3 ± 1 | 3 ± 1 | 5 ± 2 | 12 ± 1 | 7 ± 2 | 39 ± 3 | 48 ± 2 |
| YLR134W | PDC5, Pyruvate decarboxylase | 57 ± 1 | 50 ± 7 | 38 ± 10 | 95 ± 3 | 145 ± 2 | 86 ± 18 | 84 ± 6 |
| YGR087C | PDC6, Third, minor isozyme | 52 ± 2 | 146 ± 21 | 85 ± 3 | 43 ± 18 | 50 ± 21 | 10 ± 5 | 2 ± 1 |
| | of pyruvate decarhoxylase | | | | | | | |
| Acetate | | | | | | | | |
| YKR096W | ALD1, Similarity to | 84 ± 2 | 97 ± 2 | 74 ± 10 | 70 ± 3 | 92 ± 2 | 77 ± 9 | 60 ± 16 |
| | mitochondrial aldehyde | | | | | | | |
| | dehydrogenase Ald1p | | | | | | | |
| YMR170C | ALD2, Aldehyde | 36 ± 1 | 24 ± 6 | 39 ± 10 | 77 ± 12 | 12 ± 2 | 36 ± 9 | 27 ± 3 |
| | dehydrogenase, | | | | | | | |
| | (NAD(P)⁺), | | | | | | | |
| | likely cytosolic | | | | | | | |
| YMR169C | ALD3, Aldehyde | 33 ± 6 | 7 ± 1 | 9 ± 3 | 14 ± 1 | 37 ± 1 | 35 ± 17 | 31 ± 5 |
| | dehydrogenase (NAD(P)⁺) | | | | | | | |
| YOR374W | ALD4, Aldehyde dehydrogenase | 1546 ± 37 | 1680 ± 40 | 1970 ± 260 | 1883 ± 75 | 2073 ± 56 | 2474 ± 159 | 1864 ± 145 |
| YER073W | ALD5, Aldehyde | 161 ± 6 | 268 ± 10 | 243 ± 25 | 209 ± 36 | 124 ± 11 | 92 ± 5 | 89 ± 1 |
| | dehydrogenase (NAD⁺) | | | | | | | |

Genome-wide search for up- and down-regulated genes. Earlier work has been focused on comparisons between two strains, one control strain and another strain with enhanced xylose growth (Sonderegger et al., 2004a; Wahlbom et al., 2003b). These investigations revealed a large number of significantly changed genes, making it difficult to select a few candidate genes for future genetic work. We tried to overcome this problem by investigating data from several data-sets simultaneously. Since C1, C5 and BH42 originate from TMB3001, and TMB3400 originates from TMB3399 these strains make good candidates for simultaneous analysis. F12, which does not have a control strain, was not included in the analysis.

An absolute SLR-value of 1.0 (fold-change above or equal to 2.0 or below or equal to 0.5) in combination with change call I or D, was used as cut-off for selection of genes with changed expression levels. Four sets of strains were compared: C1 and BH42 versus TMB3001 metabolizing glucose and xylose, C5 on xylose versus TMB3001 on glucose and xylose, and TMB3400 on xylose versus TMB3399 on glucose. C5 growing on xylose was chosen because no cultivation with C5 on a glucose/xylose mixture was available. TMB3400 on xylose was chosen, since previous analyses with TMB3399 and TMB3400 on a glucose/xylose mixture only revealed one changed gene (YEL041W) in combination with the other strains (data not shown), indicating that most of the changes in TMB3400 were glucose-repressed, and could therefore only be observed when xylose was the sole carbon source.

No genes were down-regulated, whereas 7 genes were up-regulated in the 4 xylose-growing strains (Table 4). These genes involved YEL041W, encoding a protein which shows similarity to an NAD⁺ kinase, GAL1, GAL2, GAL7 and GAL10, encoding genes in galactose metabolism, PUT4, encoding a proline-specific permease, and the uncharacterized ORF YLR152c.

TABLE 4
Genes with enhanced expression levels (=SLR as stated, change call solely I) in BH42, C1, C5 and TMB3400 compared with the
reference strains TMB3001 and TMB3399 are shown for all strains. Enhanced genes written shown in bold and down-regulated genes are
written in italics. Strains were cultivated on glucose (g), glucose and xylose (gx) or xylose alone (x).

| | | TMB3001 | | | | | | |
|---|---|---|---|---|---|---|---|---|
| | | gx | | | | TMB3399 g | | |
| ORF | Affymetrix annotation | Ref. strain | C1 gx | C5 x | BH42 gx | Ref. strain | TMB3400 x | F12 gx |
| ALL, SLR ≧ 1 | | | | | | | | |
| YEL041W | Strong similarity to Utr1p, | 382 ± 19 | 1346 ± 98 | 1908 ± 341 | 1262 ± 125 | 49 ± 5 | 502 ± 71 | 248 ± 50 |
| | which has NAD⁺ kinase | | | | | | | |
| | activity | | | | | | | |
| YBR020W | GAL1, Galactokinase | 3 ± 1 | 1085 ± 20 | 1066 ± 181 | 57 ± 2 | 5 ± 1 | 88 ± 13 | 11 ± 1 |
| YLR081W | GAL2, Galactose permease | 8 ± 1 | 1633 ± 34 | 1651 ± 35 | 557 ± 55 | 9 ± 1 | 610 ± 77 | 26 ± 4 |
| YBR018C | GAL7, Galactose-1-phosphate | 5 ± 1 | 1188 ± 16 | 1372 ± 218 | 149 ± 37 | 9 ± 2 | 333 ± 1 | 10 ± 1 |
| | uridyl transferase | | | | | | | |
| YBR019C | GAL10, UDP-glucose | 9 ± 1 | 1062 ± 35 | 1056 ± 101 | 133 ± 13 | 8 ± 1 | 239 ± 43 | 4 ± 1 |
| | 4-epimerase | | | | | | | |
| YOR348C | PUT4, Putative proline- | 242 ± 28 | 1200 ± 89 | 1157 ± 396 | 1126 ± 124 | 523 ± 55 | 1263 ± 160 | 1013 ± 199 |
| | specific permease | | | | | | | |
| YLR152C | Similarity to YOR3165w | 157 ± 10 | 673 ± 27 | 693 ± 81 | 611 ± 69 | 184 ± 1 | 601 ± 83 | 268 ± 37 |
| C1, BH42 vs | and YNL095c | | | | | | | |
| TMB3001 | | | | | | | | |
| SLR ≧ 1.5 | | | | | | | | |

YEL041W, YBR020W, YLR081W, YBR018C, YBR019C, YOR348C, YLR152C: See data above

| | | | | | | | | |
|---|---|---|---|---|---|---|---|---|
| YDR009W | GAL3, Galactokinase | 17 ± 2 | 358 ± 8 | 295 ± 63 | 74 ± 19 | 41 ± 6 | 69 ± 7 | 65 ± 16 |
| YOR383C | FIT3, Iron transport | 111 ± 14 | 1322 ± 183 | 1355 ± 32 | 572 ± 103 | 383 ± 31 | 192 ± 10 | 1383 ± 168 |
| YOR313C | SPS4, Sporulation-specific | 6 ± 1 | 52 ± 8 | 34 ± 6 | 50 ± 20 | 11 ± 1 | 16 ± 2 | 3 ± 1 |
| | protein | | | | | | | |
| YLR439W | MRPL4, Mitochondrial | 58 ± 5 | 130 ± 19 | 159 ± 40 | 150 ± 16 | 93 ± 8 | 77 ± 26 | 92 ± 9 |
| | 60S ribosomal protein L4 | | | | | | | |
| YOL110W | SHR5, Involved in RAS | 73 ± 12 | 326 ± 18 | 269 ± 15 | 259 ± 37 | 163 ± 17 | 105 ± 10 | 277 ± 74 |
| | localization and palmitoylation | | | | | | | |
| TMB3400 vs | | | | | | | | |
| TMB3399 | | | | | | | | |
| SLR ≧ 2.5 | | | | | | | | |

YEL041W, YBR020W, YLR081W, YBR018C, YBR019C: See data above

| | | | | | | | | |
|---|---|---|---|---|---|---|---|---|
| YJR094C | IME1, meiotic gene | 23 ± 4 | 45 ± 9 | 19 ± 4 | 158 ± 116 | 5 ± 1 | 64 ± 10 | 12 ± 2 |
| | expression, meiosis | | | | | | | |
| | inducing protein | | | | | | | |
| YPL277C | strong similarity to | 30 ± 2 | 25 ± 7 | 40 ± 1 | 18 ± 1 | 12 ± 4 | 83 ± 3 | 41 ± 6 |
| | hypothetical protein | | | | | | | |
| | YOR389w/putative pseudogene | | | | | | | |
| YPL265W | DIP5, dicarboxylic amino | 76 ± 3 | 109 ± 19 | 74 ± 9 | 103 ± 84 | 76 ± 7 | 741 ± 117 | 117 ± 44 |
| | acid permease | | | | | | | |

In order to select for other mutations which may have taken place in the different strains, and would have been missed in the simultaneous comparisons, two further comparisons were made: (i) C1 and BH42 were compared to TMB3001 metabolizing glucose and xylose, and (ii) TMB3400 growing on xylose was compared to TMB3399 growing on glucose.

An absolute cut-off SLR value of 1.5 combined with change call I or D was used for selection of genes with changed expression levels in C1 and BH42. These selection criteria generated 12 up-regulated and 7 down-regulated genes (Table 4 and 5). Five of the up-regulated genes did not appear in the previous analysis: GAL3, encoding galactokinase, FIT3, encoding a protein involved in iron transport, SPS4, encoding a sporulation specific protein, MRPL4, encoding a mitochondrial ribosomal protein, and SHR5, encoding a protein involved in RAS localization and palmitoylation. A number of genes in the mating cascade were down-regulated: The MFA1 and MFA2 genes encoding mating a-factor pheromone precursors and the STE2 gene encoding an alpha-factor pheromone receptor. BAR1, encoding a protein with a-cell barrier activity, AGA2, encoding an adhesion subunit of a-agglutinin, SRD1, encoding a transcription factor, and PHO13, encoding p-nitrophenyl phosphatase were also down-regulated in C1 and BH42.

TABLE 5
Genes with decreased expression levels (=SLR as stated, change call solely D) in BH42, C1, C5 and TMB3400
compared with the
reference strains TMB3001 and TMB3399 are shown for all strains. Signals for up-regulated genes are
written in bold. Strains were cultivated
on glucose (g), glucose and xylose (gx) or xylose alone (x).

| | | TMB3001 | | | | | | |
|---|---|---|---|---|---|---|---|---|
| | | gx | C1 | | BH42 | TMB3399 g | | F12 |
| ORF | Affymetrix annotation | Ref. strain | gx | C5 x | gx | Ref. strain | TMB3400 x | gx |
| C1, BH42 | | | | | | | | |
| vs | | | | | | | | |
| TMB3001 | | | | | | | | |
| SLR ≦ −1.5 | | | | | | | | |
| YDR461W | MFA1, a-factor mating pheromone | 123 ± 8 | 7 ± 3 | 25 ± 8 | 12 ± 5 | 6 ± 2 | 13 ± 1 | 21 ± 11 |
| | precursor | | | | | | | |
| YNL145W | MFA2, a-factor mating pheromone | 826 ± 38 | 268 ± 9 | 224 ± 42 | 111 ± 18 | 30 ± 2 | 38 ± 1 | 212 ± 85 |
| | precursor | | | | | | | |
| YFL026W | STE2, Alpha-factor pheromone receptor | 109 ± 7 | 11 ± 1 | 24 ± 5 | 8 ± 1 | 5 ± 1 | 10 ± 1 | 17 ± 6 |
| YIL015W | BAR1, a-cell barrier activity on alpha | 75 ± 12 | 17 ± 2 | 40 ± 1 | 9 ± 2 | 3 ± 1 | 1 ± 1 | 8 ± 4 |
| | factor | | | | | | | |
| YGL032C | AGA2, Adhesion subunit of | 118 ± 20 | 30 ± 1 | 27 ± 5 | 20 ± 4 | 4 ± 1 | 6 ± 3 | 15 ± 5 |
| | a-agglutinin | | | | | | | |
| YCR018C | SRD1, Transcription regulator | 206 ± 23 | 61 ± 7 | 61 ± 3 | 29 ± 5 | 2 ± 1 | 3 ± 1 | 2 ± 1 |
| YDL236W | PHO13, p-nitrophenyl phosphatase | 188 ± 5 | 48 ± 4 | 43 ± 2 | 43 ± 7 | 263 ± 27 | 206 ± 11 | 340 ± 12 |
| TMB3400 | | | | | | | | |
| vs | | | | | | | | |
| TMB3399 | | | | | | | | |
| SLR ≦ −2.5 | | | | | | | | |
| YPL187W | MF(ALPHA)1, Mating factor alpha | 28 ± 3 | 31 ± 3 | 35 ± 1 | 73 ± 10 | 1335 ± 306 | 25 ± 3 | 74 ± 9 |
| YGL089C | MF(ALPHA)2, Mating factor alpha | 5 ± 1 | 5 ± 1 | 6 ± 1 | 7 ± 1 | 242 ± 11 | 9 ± 1 | 7 ± 3 |
| YBL016W | FUS3, A CDC28/CDC2 related protein | 47 ± 13 | 42 ± 2 | 33 ± 9 | 5 ± 1 | 15 ± 2 | 1 ± 1 | 4 ± 3 |
| | kinase with a positive role in | | | | | | | |
| | conjugation | | | | | | | |
| YKL178C | STE3, a factor recptor | 37 ± 5 | 14 ± 2 | 7 ± 1 | 19 ± 5 | 190 ± 14 | 20 ± 1 | 17 ± 1 |
| YLR040C | Weak similartity to hypothetical protein | 9 ± 1 | 4 ± 1 | 3 ± 1 | 5 ± 1 | 242 ± 27 | 3 ± 1 | 13 ± 5 |
| | YIL011W | | | | | | | |
| YNL335W | Similarity to Myrothecium verrucaria | 43 ± 4 | 48 ± 9 | 13 ± 1 | 80 ± 2 | 421 ± 22 | 7 ± 1 | 24 ± 7 |
| | cyanamide hydratase, identical to | | | | | | | |
| | hypothetical protein YFL061w | | | | | | | |
| YNR064C | Similarity to Rhodobacter capsulatus 1- | 4 ± 1 | 2 ± 1 | 2 ± 1 | 8 ± 3 | 135 ± 35 | 7 ± 1 | 2 ± 1 |
| | chloroalkane halidohydrolase | | | | | | | |
| YOR237W | HES1, homology to human oxysterol | 9 ± 1 | 10 ± 1 | 11 ± 6 | 11 ± 8 | 21 ± 9 | 1 ± 1 | 3 ± 1 |
| | binding protein | | | | | | | |

An absolute SLR value of 2.5 combined with change call I or D was used for selection of genes with changed expression levels in TMB3400 compared to TMB3399. A higher SLR-value was chosen to limit the number of candidate genes from this strain to strain comparison. The comparison yielded 8 up-regulated and 8 down-regulated genes (Table 4 and 5). Only 3 of the up-regulated genes did not occur in the comparison including all strains: IME1, encoding a protein involved in meiotic gene expression, the uncharacterized ORF YPL277C and DIPS, encoding an amino acid permease. Here again, several genes involved in mating were down-regulated in TMB3400, however, it was another set of genes than what was found for BH42 and C1: MF(ALPHA)1 and MF(ALPHA)₂, encoding alpha mating factors, FUS3, encoding a CDC28/CDC2 related protein kinase, and STE3, encoding an a-factor receptor. Three uncharacterized ORFs, YLR040C, YNL335W and YNR064C, as well as HES1, encoding a protein similar to human oxysterol binding protein, were also down-regulated in TMB3400.

### Small Changes Observed in all Strains at Several Conditions Simultaneously.

In the previous analysis (Table 4 and 5) both SLR- and p-value was used for selection of genes with changed expression levels. However, genes with a low absolute SLR-value can still have a high likelihood of being changed. All strains were therefore included in different comparisons using change call I or D as cut-off. Unlike previous analyses, the comparisons also included anaerobic cultivations of C1 and TMB3001, as well as xylose cultivation with C1: (i) C1 and BH42 versus TMB3001 utilizing glucose/xylose aerobically, (ii) C1 versus TMB3001 utilizing glucose/xylose anaerobically, (iii) C1 and C5 utilizing xylose versus TMB3001 utilizing glucose/xylose aerobically and (iv) TMB3400 utilizing xylose versus TMB3399 utilizing glucose aerobically and (v) TMB3400 versus TMB3399 utilizing glucose and glucose/xylose aerobically (Table 6). Three genes resulted from these comparisons: SOL3, encoding a protein with similarities to glucose-6-phosphate dehydrogenase, and YEL041W, encoding a protein with possible NAD⁺ kinase activity, were up-regulated, whereas the uncharacterized ORF YLR042C was down-regulated. When the fifth comparison was disregarded (TMB3400 versus TMB3399 utilizing glucose and glucose/xylose), two more down-regulated and 9 more up-regulated ORFs were identified. GAL1, GAL2, GAL7 and GAL10 in the galactose metabolism were up-regulated. Also the PPP gene TAL1, the PUT4 gene encoding a putative proline permease, and the HISS gene encoding imidazoleglycerol phosphate dehydratase, were up-regulated. The uncharacterized ORF YIL110W, as well as RPA49, encoding the alpha subunit of RNA polymerase A, were down-regulated, whereas the uncharacterized ORF YLR152c was up-regulated. Most of the genes with changed expression levels were also found when selecting for certain SLR-values (Table 4 and 5), with the exception of SOL3, TAL1, HIS3, RPA49, YLR042C and YIL110W which were only identified when using change call I or D as cut-off.

TABLE 6
Signals for genes with changed expression level (change call solely D (italic) or I (bold))
when all xylose growing strains were compared with their reference strains (BH42, C1 and C5
versus TMB3001, TMB3400 versus TMB3399). Strains were cultivated on glucose (g), glucose
and xylose (gx) or xylose alone (x) under aerobic (aer) or anerobic (ana) conditions.

| | | 3001 | | | |
|---|---|---|---|---|---|
| | | gx aer | 3001 | | |
| | | Ref. | gx ana | C1 | C1 |
| ORF | Affymetrix annotation | strain | Ref. strain | gx aer | gx ana |
| YHR163W | SOL3, Shows similarity to | 496 ± 87 | 437 ± 53 | 1068 ± 45 | 1129 ± 17 |
| | glucose-6-phosphate | | | | |
| | dehydrogenase non-catalytic | | | | |
| | domains, homologous to Sol2p | | | | |
| | and Sol1p | | | | |
| YEL041W | Strong similarity to Utr1p, | 382 ± 19 | 39 ± 5 | 1346 ± 98 | 998 ± 119 |
| | which has NAD⁺ kinase activity | | | | |
| YBR020W | GAL1, Galactokinase | 3 ± 1 | 6 ± 1 | 1085 ± 20 | 790 ± 101 |
| YLR081W | GAL2, Galactose permease | 8 ± 1 | 6 ± 1 | 1633 ± 34 | 1879 ± 158 |
| YBR018C | GAL7, Galactose-1-phosphate | 5 ± 1 | 4 ± 1 | 1188 ± 16 | 979 ± 179 |
| | uridyl transferase | | | | |
| YBR019C | GAL10, UDP-glucose 4- | 9 ± 1 | 13 ± 3 | 1062 ± 35 | 846 ± 53 |
| | epimerase | | | | |
| YLR354C | TAL1, Transaldolase, enzyme | 380 ± 29 | 409 ± 47 | 684 ± 46 | 980 ± 52 |
| | in the pentose phosphate | | | | |
| | pathway | | | | |
| YOR348C | PUT4, Putative proline-specific | 242 ± 28 | 28 ± 6 | 1200 ± 89 | 95 ± 27 |
| | permease | | | | |
| YOR202W | HIS3, Imidazoleglycerol- | 346 ± 15 | 244 ± 14 | 834 ± 13 | 580 ± 16 |
| | phosphate dehydratase | | | | |
| YLR152C | Similarity to YOR3165w and | 157 ± 10 | 165 ± 4 | 673 ± 27 | 787 ± 34 |
| | YNL095c | | | | |
| YLR042C | hypothetical protein | 33 ± 3 | 34 ± 6 | 7 ± 1 | 6 ± 1 |
| YIL110W | Weak similarity to hypothetical | 70 ± 3 | 59 ± 8 | 42 ± 5 | 33 ± 5 |
| | Caenorhabditis elegans protein | | | | |
| YNL248C | RPA49, 49-kDa alpha subunit | 136 ± 3 | 128 ± 1 | 96 ± 4 | 101 ± 5 |
| | of RNA polymerase A | | | | |

| | | | | 3399 g | |
|---|---|---|---|---|---|
| | C1 | C5 | BH42 | Ref. | |
| ORF | x aer | x aer | gx aer | strain | 3400 x |
| YHR163W | 1285 ± 211 | 1121 ± 36 | 1072 ± 109 | 600 ± 114 | 1262 ± 16 |
| YEL041W | 1514 ± 215 | 1908 ± 341 | 1262 ± 125 | 49 ± 5 | 502 ± 71 |
| YBR020W | 808 ± 234 | 1066 ± 181 | 57 ± 2 | 5 ± 1 | 88 ± 13 |
| YLR081W | 2206 ± 274 | 1651 ± 35 | 557 ± 55 | 9 ± 1 | 610 ± 77 |
| YBR018C | 1301 ± 372 | 1372 ± 218 | 149 ± 37 | 9 ± 2 | 333 ± 1 |
| YBR019C | 775 ± 103 | 1056 ± 101 | 133 ± 13 | 8 ± 1 | 239 ± 43 |
| YLR354C | 875 ± 161 | 845 ± 11 | 745 ± 54 | 509 ± 50 | 825 ± 4 |
| YOR348C | 1604 ± 428 | 1157 ± 396 | 1126 ± 124 | 523 ± 55 | 1263 ± 160 |
| YOR202W | 870 ± 18 | 990 ± 123 | 500 ± 3 | 267 ± 17 | 412 ± 41 |
| YLR152C | 810 ± 12 | 693 ± 81 | 611 ± 69 | 184 ± 1 | 601 ± 83 |
| YLR042C | 3 ± 2 | 14 ± 4 | 20 ± 11 | 38 ± 5 | 12 ± 3 |
| YIL110W | 22 ± 10 | 38 ± 12 | 25 ± 2 | 72 ± 15 | 34 ± 7 |
| YNL248C | 81 ± 15 | 85 ± 1 | 70 ± 4 | 129 ± 13 | 77 ± 2 |

TABLE 7
Signals from ORFs representing genes with changed expression levels (=Change call solely I or D
in at least one strain compared with its reference strain) involved in the galactose metabolism are
shown. Up-regulated genes are shown in bold and down-regulated genes in italic. Strains were cultivated
on glucose (g), glucose and xylose (gx) or xylose alone (x).

| | | TMB3001 | | | | | |
|---|---|---|---|---|---|---|---|
| | | gx | | | | | |
| | | Ref. | C1 | | | | BH42 |
| ORF | Annotation | strain | gx | C1 x | C5 x | BH42 g | gx |
| YBR020W | GAL1, | 3 ± 1 | 1085 ± 20 | 808 ± 234 | 1066 ± 181 | 12 ± 5 | 57 ± 2 |
| | Galactokinase | | | | | | |
| YLR081W | GAL2, Galactose | 8 ± 1 | 1633 ± 34 | 2206 ± 274 | 1651 ± 35 | 65 ± 6 | 557 ± 55 |
| | permease | | | | | | |
| YMR105C | GAL5, | 120 ± 56 | 471 ± 35 | 576 ± 59 | 421 ± 13 | 429 ± 16 | 394 ± 50 |
| | Phosphoglucomutase | | | | | | |
| YBR018C | GAL7, Galactose- | 5 ± 1 | 1188 ± 16 | 1301 ± 372 | 1372 ± 218 | 22 ± 3 | 149 ± 37 |
| | 1-phosphate uridyl | | | | | | |
| | transferase | | | | | | |
| YBR019C | GAL10, UDP- | 9 ± 1 | 1062 ± 35 | 775 ± 103 | 1056 ± 101 | 22 ± 3 | 133 ± 13 |
| | glucose 4- | | | | | | |
| | epimerase | | | | | | |
| YDR009W | GAL3, | 17 ± 2 | 358 ± 8 | 248 ± 114 | 295 ± 63 | 59 ± 6 | 74 ± 19 |
| | Galactokinase | | | | | | |
| YML051W | GAL80, Regulatory | 55 ± 1 | 223 ± 9 | 190 ± 107 | 177 ± 47 | 101 ± 10 | 118 ± 15 |
| | protein | | | | | | |
| YNL239W | GAL6, | 132 ± 13 | 343 ± 9 | 358 ± 12 | 286 ± 25 | 270 ± 24 | 284 ± 29 |
| | Aminopeptidase of | | | | | | |
| | cysteine protease | | | | | | |
| | family | | | | | | |
| YOL051W | GAL11, | 100 ± 5 | 215 ± 17 | 287 ± 44 | 228 ± 10 | 80 ± 12 | 90 ± 13 |
| | Component of the | | | | | | |
| | RNA polymerase | | | | | | |
| | II holoenzyme | | | | | | |
| | complex, positive | | | | | | |
| | and negative | | | | | | |
| | transcriptional | | | | | | |
| | regulator of genes | | | | | | |
| | involved in mating- | | | | | | |
| | type specialization | | | | | | |
| YPL248C | GAL4, GAL81, | 26 ± 7 | 38 ± 4 | 55 ± 7 | 27 ± 1 | 57 ± 8 | 62 ± 15 |
| | zinc-finger | | | | | | |
| | transcription | | | | | | |
| | factor of the Zn(2)- | | | | | | |
| | Cys(6) binuclear | | | | | | |
| | cluster domain | | | | | | |
| | type | | | | | | |
| YHR193C | GAL4 enhancer | 828 ± 39 | 581 ± 40 | 542 ± 49 | 595 ± 6 | 752 ± 46 | 607 ± 30 |
| | protein, homolog | | | | | | |
| | of human alpha | | | | | | |
| | NAC subunit of | | | | | | |
| | the nascent- | | | | | | |
| | polypeptide- | | | | | | |
| | associated complex | | | | | | |
| YLR071C | Component of | 61 ± 1 | 94 ± 7 | 85 ± 34 | 63 ± 12 | 77 ± 4 | 81 ± 1 |
| | RNA polymerase | | | | | | |
| | II | | | | | | |
| | holoenzyme/\mediator | | | | | | |
| | complex, | | | | | | |
| | interacts with | | | | | | |
| | Sin4p, Gal11p, and | | | | | | |
| | a 50 kd | | | | | | |
| | polypeptide | | | | | | |

| | | | | TMB3399 | | |
|---|---|---|---|---|---|---|
| | | | TMB3399 g | gx | | |
| | | F12 | Ref. | Ref. | TMB3400 | |
| ORF | F12 g | gx | strain | strain | gx | TMB3400 x |
| YBR020W | 8 ± 1 | 11 ± 1 | 5 ± 1 | 6 ± 2 | 7 ± 1 | 88 ± 13 |
| YLR081W | 7 ± 1 | 26 ± 4 | 9 ± 1 | 9 ± 1 | 6 ± 4 | 610 ± 77 |
| YMR105C | 602 ± 129 | 491 ± 43 | 342 ± 25 | 354 ± 146 | 322 ± 76 | 729 ± 98 |
| YBR018C | 7 ± 2 | 10 ± 1 | 9 ± 2 | 10 ± 1 | 14 ± 1 | 333 ± 1 |
| YBR019C | 4 ± 1 | 4 ± 1 | 8 ± 1 | 11 ± 2 | 8 ± 1 | 239 ± 43 |
| YDR009W | 39 ± 8 | 65 ± 16 | 41 ± 6 | 46 ± 2 | 38 ± 8 | 69 ± 7 |
| YML051W | 102 ± 1 | 135 ± 4 | 119 ± 2 | 120 ± 5 | 99 ± 6 | 100 ± 1 |
| YNL239W | 236 ± 3 | 239 ± 20 | 230 ± 16 | 294 ± 8 | 197 ± 15 | 179 ± 3 |
| YOL051W | 135 ± 12 | 118 ± 1 | 100 ± 11 | 132 ± 54 | 144 ± 35 | 171 ± 10 |
| YPL248C | 77 ± 6 | 81 ± 2 | 30 ± 5 | 28 ± 3 | 33 ± 4 | 26 ± 5 |
| YHR193C | 887 ± 94 | 821 ± 57 | 885 ± 123 | 799 ± 135 | 799 ± 123 | 865 ± 6 |
| YLR071C | 74 ± 3 | 81 ± 5 | 46 ± 9 | 41 ± 8 | 43 ± 10 | 63 ± 2 |

TABLE 8
Signals from ORFs representing genes with changed expression level (=Change call I or D in at least one strain compared with its
reference strain) in the mating pathway. Signals for up-regulated genes are written in bold, and signals for down-regulated genes are written in
italic. Strains were cultivated on glucose (g), glucose and xylose (gx) or xylose alone (x).

| | | TMB3001 | | | | | | |
|---|---|---|---|---|---|---|---|---|
| | | gx | | | | | TMB3399 g | |
| | | Ref. | | | | | Ref. | |
| ORF | Annotation | strain | C1 gx | C5 x | BH42 gx | F12 gx | strain | TMB3400 x |
| YDR461W | MFA1, a-factor mating pheromone precursor | 123 ± 8 | 7 ± 3 | 25 ± 8 | 12 ± 5 | 21 ± 11 | 6 ± 2 | 13 ± 1 |
| YNL145W | MFA2, a-factor mating pheromone precursor | 826 ± 38 | 268 ± 9 | 224 ± 42 | 111 ± 18 | 212 ± 85 | 30 ± 2 | 38 ± 1 |
| YFL026W | STE2, Alpha-factor pheromone receptor | 109 ± 7 | 11 ± 1 | 24 ± 5 | 8 ± 1 | 17 ± 6 | 5 ± 1 | 10 ± 1 |
| YOR212W | STE4, beta subunit of G protein coupled | 254 ± 3 | 163 ± 5 | 188 ± 2 | 85 ± 13 | 64 ± 11 | 112 ± 12 | 118 ± 17 |
| | to mating factor receptor | | | | | | | |
| YJR086W | STE18, gamma subunit of G protein | 86 ± 11 | 44 ± 5 | 27 ± 2 | 4 ± 1 | 8 ± 2 | 14 ± 2 | 3 ± 1 |
| | coupled to mating factor receptors | | | | | | | |
| YGR040W | KSS1, MAP protein kinase homolog | 19 ± 3 | 12 ± 1 | 18 ± 4 | 7 ± 1 | 6 ± 2 | 7 ± 2 | 5 ± 1 |
| | involved in pheromone signal transduction | | | | | | | |
| YLR265C | NEJ1, hypothetical protein | 20 ± 4 | 9 ± 2 | 5 ± 2 | 1 ± 1 | 0 | 1 ± 1 | 0 |
| YPL187W | MF(ALPHA)1, Mating factor alpha | 28 ± 3 | 31 ± 3 | 35 ± 1 | 73 ± 10 | 74 ± 9 | 1335 ± 306 | 25 ± 3 |
| YGL089C | MF(ALPHA)2, Mating factor alpha | 5 ± 1 | 5 ± 1 | 6 ± 1 | 7 ± 1 | 7 ± 3 | 242 ± 11 | 9 ± 1 |
| YKL178C | STE3, a factor receptor | 37 ± 5 | 14 ± 2 | 7 ± 1 | 19 ± 5 | 17 ± 1 | 190 ± 14 | 20 ± 1 |
| YBL016W | FUS3, a CDC28/CDC2 related protein | 47 ± 13 | 42 ± 2 | 33 ± 9 | 5 ± 1 | 4 ± 3 | 15 ± 2 | 1 ± 1 |
| | kinase with a positive role in conjugation | | | | | | | |
| YOL051W | GAL11, Component of the RNA | 100 ± 5 | 215 ± 17 | 228 ± 10 | 90 ± 13 | 118 ± 1 | 100 ± 11 | 171 ± 10 |
| | polymerase II holoenzyme complex, | | | | | | | |
| | positive and negative transcriptional | | | | | | | |
| | regulator of genes involved in | | | | | | | |
| | mating-type specialization | | | | | | | |
| YHR084W | STE12, Transcription factor | 34 ± 1 | 26 ± 2 | 36 ± 4 | 17 ± 1 | 21 ± 1 | 28 ± 1 | 16 ± 1 |
| YDR103W | STE5, Protein of the pheromone pathway | 43 ± 2 | 45 ± 2 | 48 ± 9 | 14 ± 1 | 18 ± 1 | 12 ± 1 | 11 ± 3 |
| YHR005C | GPA1, alpha subunit of G protein coupled to | 40 ± 3 | 31 ± 1 | 34 ± 4 | 5 ± 1 | 7 ± 1 | 9 ± 2 | 4 ± 1 |
| | mating factor receptors | | | | | | | |

The structural genes GAL1, GAL2, GAL5, GAL7 and GAL10 were up-regulated in C1, C5, BH42 and TMB3400 compared to TMB3001 and TMB3399 (Table 7). The regulatory genes GAL3 and GAL80, as well as GAL6 were up-regulated in C1, C5 and BH42. GAL11 was enhanced in C1, C5 and TMB3400. F12 had comparatively high levels of GAL2, GAL5, GAL3, GAL80, GAL6 and GAL4. TMB3399 had high levels of GAL3, GAL6 and GAL80 compared to TMB3001, which might explain why these genes were not enhanced in TMB3400. Thus genes involved in the galactose metabolism were up-regulated for the xylose growing S. cerevisiae strains C1, C5, BH42 and TMB3400, and several GAL genes have a high expression in F12. Several genes in the galactose metabolism were induced by xylose in the presence of glucose in BH42 (Table 7). However, in TMB3400 GAL gene expression was enhanced only when xylose was present and glucose was absent.

Out of 19 genes involved in mating (Saccharomyces Genome Database (SGD); Elion, 2000), the expression level of 15 genes was changed in at least one of the xylose growing strains (Table 8). Generally the genes were down-regulated, with the exception of GAL11, encoding a transcriptional regulator of genes involved in mating type specialization, which was up-regulated in C1, C5 and TMB3400.

MFA1 and MFA2, encoding mating a-factor pheromone precursors, were down-regulated in C1, C5, and BH42 and comparatively low in TMB3399, TMB3400 and F12. This was also observed for STE2, encoding an alpha-factor receptor, and STE4 and STE18, encoding the beta- and gamma-subunit, respectively, of the G protein coupled to mating factor receptor. Also KSS1, encoding a protein involved in pheromone signal transduction, was down-regulated in C1 and BH42, and NEJ1 was down-regulated in C1, C5 and BH42 while their level was low in F12, TMB3399 and TMB3400. MF(ALPHA)1 and MF(ALPHA)₂ genes, encoding mating alpha factors, and STE3, encoding the a-factor receptor were only down-regulated in TMB3400, but their expression levels were comparatively low in all other strains. FUS3, encoding a CDC28/CDC2 related protein kinase, was down-regulated in BH42 and TMB3400, and expressed at low level in F12. STE12, encoding a transcription factor, STE5, encoding a protein of the pheromone pathway, and GPA1, encoding the alpha subunit of the G-protein coupled to mating factor receptors, were down-regulated in BH42 only.

### Transcription Regulators.

The expression levels of transcription regulators were investigated since they can regulate transcription of a whole set of genes by binding a promoter or an enhancer DNA sequence or interact with a DNA-binding transcription factor. The SGD and Affymetrix annotations were screened for the word “transcription” and the expression level of all resulting genes was investigated. BH42 and C1 utilizing glucose/xylose and C5 utilizing xylose were compared to TMB3001 utilizing glucose/xylose. TMB3400 utilizing xylose was compared to TMB3399 utilizing glucose. No transcriptional regulators were changed in all strains, and therefore change call solely I or D in three out of four strains was used as cut-off (Table 9).

TABLE 9
Signals for changed transcription regulators (=change call solely I or D in at least three out of four strains when compared to their
reference strains). Signals for up-regulated genes are written in bold, and signals for down-regulated genes are written in italic. Strains were
cultivated on glucose (g), glucose and xylose (gx) or xylose alone (x).

| | | TMB3001 | | | | | | |
|---|---|---|---|---|---|---|---|---|
| | | gx | | | | | TMB3399 g | |
| | | Ref. | | | | | Ref. | |
| ORF | Annotation | strain | C1 gx | C5 x | BH42 gx | F12 gx | strain | TMB3400 x |
| YOR230W | WTM1, Transcriptional modulator: | 602 ± 111 | 1060 ± 31 | 1156 ± 5 | 1328 ± 8 | 1430 ± 72 | 1314 ± 169 | 768 ± 44 |
| | meiotic regulation | | | | | | | |
| YOL051W | GAL11, Component of the RNA | 100 ± 5 | 215 ± 17 | 228 ± 10 | 90 ± 13 | 118 ± 1 | 100 ± 11 | 171 ± 10 |
| | polymerase II holoenzyme | | | | | | | |
| | complex, positive and | | | | | | | |
| | negative transcriptional | | | | | | | |
| | regulator of genes involved in | | | | | | | |
| | mating-type specialization | | | | | | | |
| YIL154C | IMP2, Transcription factor: | 15 ± 1 | 90 ± 10 | 69 ± 15 | 35 ± 3 | 55 ± 9 | 60 ± 12 | 70 ± 5 |
| | Protein involved in | | | | | | | |
| | nucleo-mitochondrial | | | | | | | |
| | control of maltose, galactose and | | | | | | | |
| | raffinose utilization, activates | | | | | | | |
| | transcription from RNApol II | | | | | | | |
| | promoter | | | | | | | |
| YML051W | GAL80, Regulatory protein, | 55 ± 1 | 223 ± 9 | 177 ± 47 | 118 ± 15 | 135 ± 4 | 119 ± 2 | 100 ± 1 |
| | inhibits transcription activation | | | | | | | |
| | by Gal4p in the absence | | | | | | | |
| | of galactose. | | | | | | | |
| YBL066C | SEF1, Putative transcription factor | 14 ± 1 | 29 ± 3 | 27 ± 4 | 31 ± 4 | 31 ± 2 | 17 ± 3 | 23 ± 3 |
| YFL021W | GAT1, Transcriptional activator | 81 ± 24 | 234 ± 11 | 310 ± 45 | 231 ± 10 | 172 ± 16 | 58 ± 11 | 38 ± 6 |
| | with GATA-1-type Zn finger | | | | | | | |
| | DNA-binding motif: activator | | | | | | | |
| | of transcription of | | | | | | | |
| | nitrogen-regulated genes, | | | | | | | |
| | RNApol II transcription factor | | | | | | | |
| | activitiy | | | | | | | |
| YOR290C | SNF2, Transcriptional regulator, | 63 ± 1 | 324 ± 57 | 154 ± 29 | 123 ± 1 | 66 ± 11 | 52 ± 5 | 74 ± 3 |
| | regulation of phospholipid | | | | | | | |
| | synthesis | | | | | | | |
| YGR067C | Weak similarity to transcription | 206 ± 37 | 325 ± 36 | 227 ± 20 | 373 ± 60 | 441 ± 26 | 140 ± 8 | 395 ± 35 |
| | factors | | | | | | | |
| YHR206W | SKN7, Protein with similarity to | 57 ± 2 | 115 ± 3 | 113 ± 13 | 90 ± 8 | 94 ± 3 | 60 ± 15 | 33 ± 2 |
| | DNA-binding region of heat shock | | | | | | | |
| | transcription factors | | | | | | | |
| YOL116W | MSN1, 43 kDa protein, | 28 ± 2 | 65 ± 4 | 68 ± 12 | 35 ± 3 | 28 ± 1 | 21 ± 1 | 44 ± 5 |
| | transcriptional activator | | | | | | | |
| YOR363C | PIP2, Activator of peroxisome | 49 ± 3 | 84 ± 7 | 81 ± 6 | 91 ± 5 | 61 ± 5 | 39 ± 1 | 52 ± 4 |
| | proliferation | | | | | | | |
| YCL055W | KAR4, May assist Ste12p in | 44 ± 3 | 25 ± 2 | 23 ± 1 | 15 ± 1 | 17 ± 1 | 17 ± 3 | 21 ± 1 |
| | pheromone-dependent expression | | | | | | | |
| | of KAR3 and CIK1 | | | | | | | |
| YCR018C | SRD1, Transcription regulator: | 206 ± 23 | 61 ± 7 | 61 ± 3 | 30 ± 6 | 2 ± 1 | 2 ± 1 | 3 ± 1 |
| | processing of pre-rRNA to | | | | | | | |
| | mature rRNA | | | | | | | |
| YDR397C | NCB2, Repressor of class II | 89 ± 2 | 47 ± 2 | 54 ± 10 | 42 ± 10 | 52 ± 3 | 100 ± 10 | 96 ± 15 |
| | transcription | | | | | | | |

Among the 14 selected genes, three were involved in mating and two were involved in control of sugar utilisation, WTM1 involved in melotic regulation was up-regulated in C1, C5 and BH42. The transcript level of WTM1 was high in TMB3399 and F12. The GAL11 gene, involved in regulation of genes in mating type specialization, was up-regulated in C1, C5 and TMB3400. KAR4 encodes a protein that may assist Ste12p in pheromone-dependent expression of KAR3 and CIK1, and it was down-regulated in C1, C5 and BH42 and comparably low in F12 and TMB3399. The IMP2 gene, encoding a protein involved in nucleo-mitochondrial control of maltose, galactose and raffinose utilization, was up-regulated in C1, C5 and BH42 compared to TMB3001, and its expression level was high in TMB3399 and F12. GAL80, which encodes a protein that inhibits transcription activation by Gal4p in the absence of galactose (Lohr et al., 1995), was also up-regulated in C1, C5 and BH42, and it was comparably high in F12 and TMB3399.

Genome-wide transcriptional analysis is a powerful method to identify S. cerevisiae genes whose levels have been affected by environmental or genetic changes and is therefore increasingly used as an analytical tool in metabolic engineering. However, a single comparison between a control and a modified strain or between different cultivation conditions usually reveals hundreds of genes whose level has changed, notably when the modifications affect growth. The outcome of this method is therefore limited by the tremendous amount of genes whose effect needs to be checked afterwards in order to distinguish “true” changes. Our genome-wide transcriptional analysis investigation took advantage of the occurrence of several S. cerevisiae recombinant strains that had recently been independently developed for xylose growth using different methods of strain transformation and selection (for F12: Sonderegger et al., 2004b), mutagenesis (for TMB3400: Wahlbom et al., 2003a), adaptation (for C1 and C5: Sonderegger and Sauer, 2003) and breeding (for BH42: Spencer-Martins, 2003). A simple hypothesis was used: the more strains, the less the number of false positives and the easier the identification of truly required genetic changes for efficient xylose growth.

The low xylose consumption rate and the absence of anaerobic xylose growth in recombinant xylose-utilizing S. cerevisiae strains (Eliasson et al., 2000b) might result from limitations in (i) xylose transport, because of lower affinity for xylose than for glucose (Kötter and Ciriacy, 1993), (ii) xylose pathway level (Jeppsson et al., 2003b), and (iii) PPP level (Kötter and Ciriacy, 1993), and/or from (iv) cofactor imbalance in the xylose pathway (Bruinenberg et al., 1983; Kötter and Ciriacy, 1993). The present investigation showed that enhanced xylose growth in recombinant S. cerevisiae strains was notably associated with high galactose transporter level, up-regulated PPP and galactose metabolism and down-regulated mating-metabolism. It also identified several new candidate genes, among which an NAD⁺-kinase homologue and several transcriptional regulators.

### Xylose Transport.

Gal2p, which together with Hxt4p, Hxt5p and Hxt7p, is capable of transporting xylose (via facilitated diffusion, (Busturia and Lagunas, 1986)) in S. cerevisiae (Hamacher et al., 2002), was up-regulated in all xylose-growing strains. GAL2 and HXT16 in C1 and C5, were the only up-regulated hexose transporters.

Contradictory results have previously been reported regarding the role of xylose transport and GAL2 level with respect to the limited xylose-utilization by recombinant S. cerevisiae. The low affinity of the hexose transporters for xylose (Kötter and Ciriacy, 1993) might limit xylose consumption rate. On the other hand, the calculated flux control coefficient indicated that transport only limited the xylose consumption rate at low xylose concentrations (Gárdonyl et al., 2003). Similarly over-expression of GAL2 alone did not enhance xylose growth (Hamacher et al., 2002) but a recombinant strain overexpressing the arabinose pathway grew slightly faster on arabinose when GAL2 was overexpressed (Becker and Boles, 2003). By overexpression of the S. cerevisiae GAL2 gene, a Kluyveromyces lactis strain capable of galactose growth in the absence of respiration was obtained (Goffrini et al., 2002). In our study, the highest GAL2 mRNA expression was found in C1, which is the only strain capable of anaerobic growth on xylose (Sonderegger and Sauer, 2003), (Table 7). Taken together these results suggest that GAL2 overexpression could be a necessary trait, although not sufficient, for high xylose-utilization.

Gal2p is usually inactivated by glucose at two levels, first by repression of GAL2 gene transcription and second, at the post-translational level by glucose induced inactivation, Gal4p, which activates transcription of GAL2 (and GAL1, GAL7, GAL10, MEL1) (Johnston, 1987), is itself repressed by binding of Mig1p in the presence of glucose (Nehlin et al., 1991). However, no change was observed in MIG1 mRNA level for any of the xylose-growing strains compared to their control strains (data not shown). At the protein level, Gal2p is delivered from the plasma membrane to the vacuole by endocytosis, and further degraded by vacuolar proteinases (Horak and Wolf, 1997). During glucose inactivation, the galactose transporter is ubiquinated (Horak and Wolf, 1997) through the Ubc1p-Ubc4p-Ubc5p triad of ubiquitin-conjugating enzymes and Npi1/Rsp5p ubiquitin-protein ligase (Horak and Wolf, 2001). Furthermore, the HXK2 gene product plays a role in the induction of proteolysis of Gal2p (Horak et al., 2002). Our results show that (i) END3 and END4 genes, needed for endocytosis, were down-regulated in BH42, (ii) UBC1, whose deletion enhances the half-life of Gal2p (Horak and Wolf, 2001), was down-regulated in C1, C5 and BH42, and (iii) HXK2, whose deletion abolishes Gal2p degradation, was down-regulated in TMB3400 (data not shown), and suggest that a combination of up-regulated GAL2 and impaired Gal2p inactivation improve xylose growth.

### Galactose Metabolism.

Not only the galactose transporter but most of the genes encoding the galactose pathway were up-regulated in the xylose-growing strains. C1 and BH42 displayed enhanced expression of genes in galactose metabolism when grown on a mixture on glucose and xylose, whereas the galactose metabolism was up-regulated only in the absence of glucose in TMB3400. The difference in GAL gene expression of xylose-growing strains utilizing different carbon-sources indicates that different mutations have taken place. However, all strains display enhanced expression of GAL genes when xylose is present in the medium. The GAL gene family consists of the structural genes GAL1, GAL2, GAL5, GAL7, GAL10 and MEL1, and the regulatory genes GAL3, GAL4 and GAL80 (Johnston, 1987; Lohr et al., 1995). Among the regulatory genes GAL3 and GAL80 were up-regulated in BH42, C1 and C5, and GAL4 was up-regulated in C1 on xylose (Table 7). The IMP2 gene, encoding a protein involved in nucleo-mitochondrial control of maltose, galactose and raffinose utilization (Donnini et al., 1992) was up-regulated in C1, C5 and BH42 (Table 9). In a recent investigation, Imp2p was shown to positively affect glucose derepression of Leloir pathway genes as well as the activator GAL4 (Alberti et al., 2003). Hence, an up-regulated IMP2 might be involved in the up-regulated GAL metabolism.

It is unclear why up-regulation of the whole galactose pathway would improve xylose growth. It even seems that a constitutively up-regulated galactose pathway may impair galactose growth for TMB3400 (Cronwright, 2002). The alpha-forms of D-xylose and D-galactose have similar three-dimensional structure, which might explain a role of galactose genes for xylose metabolism. Our suggestion is that the whole pathway deregulation enables the up-regulation of the galactose transporter gene GAL2, which could be the only galactose gene needed for improving xylose growth.

### Xylose Pathway.

Slow xylose utilization can be attributed to limiting levels of the introduced xylose pathway enzymes XR and XDH. Increasing the XR-activity in TMB3001 strain indeed enhanced the xylose consumption rate in oxygen-limited xylose batch culture (Jeppsson et al., 2003b). Enhanced XR and XDH enzyme activities were found in C1 and TMB3400, compared to TMB3001 and TMB3399, respectively (Sonderegger et al. 2004b; Wahlbom et al. 2003a). However, BH42 and C5 had the same enzyme activities as TMB3001, showing that enhanced XR- and XDH-activities are not necessary for enhanced xylose growth. Indeed the only modifications that we observed for the endogenous XR and XDH activities were (i) that BH42 that had a high expression level of the endogenous XYL2 gene, and (ii) that F12 that had a comparatively high expression level of GRE3, encoding an NADPH-dependent aldose reductase (Kuhn et al., 1995; Träff et al., 2002).

### Xylulokinase.

Overexpression of the endogenous xylulokinase gene has been shown to be necessary for enhancing the xylulose (Eliasson et al., 2000a; Lee et al., 2003) and the xylose (Toivari et al., 2001) fermentation rate in S. cerevisiae, but very high XK-activity (28-36 U/mg) had a negative effect on the xylose consumption rate (Johansson et al., 2001). XKS1 mRNA expression was enhanced in C1 and C5. However, the xylose growing strains, BH42 and F12 had approximately the same XKS1 expression level as TMB3001, showing that higher XK-activity was not crucial for xylose growth.

### NAD(P)H-NAD(P)⁺ Availability.

Xylitol formation in recombinant XR-XDH strains results from the cofactor imbalance caused by NAD(P)H-dependent XR in combination with NAD⁺-dependent XDH (Bruinenberg et al., 1983; Kötter and Ciriacy, 1993). Xylitol formation might be restrained if the xylose consumption rate could be enhanced, through a better regeneration of NADPH and NAD⁺ in other parts of the metabolism. Genes in the NADPH-producing oxidative pentose phosphate pathway, GND1 and SOL3, were up-regulated in BH42, C1, C5 and TMB3400, and the ZWF1 gene was up-regulated in BH42, C1 and C5. The expression level of the oxidative PPP gene ZWF1 has been shown to correlate with the xylose consumption rate at low ZWF1 expression levels (Jeppsson et al., 2003a). A metabolic flux model indicated that high specific xylose consumption rate was accompanied with high PPP flux (Wahlbom et al., 2001). The expression levels of GPD1 or GPD2 genes, encoding the NADH-dependent glycerol-3-phosphate dehydrogenase, were enhanced in several xylose-growing strains, and this may help to provide more NAD⁺ for the XDH reaction.

YEL041, which shows similarities to UTR1 was up-regulated in all the xylose-growing S. cerevisiae strains. UTR1 encodes a cytosolic NAD⁺-kinase that enables the phosphorylation of NAD⁺ to NADP⁺ (Kawai et al., 2001) and it is highly probable that the enhanced expression of YEL041W affect the amounts of cofactors available for the XR and XDH reactions.

### Pentose Phosphate Pathway.

Limitations of the PPP metabolism (Kötter and Ciriacy, 1993) could also cause limited xylose consumption rate. The over-expression of the non-oxidative PPP genes was shown to enhance the xylulose consumption rate in recombinant S. cerevisiae (Johansson and Hahn-Hägerdal, 2002). Enhanced transaldolase activity enhanced xylose growth in a plasmid strain over-expressing XYL1 and XYL2 (Walfridsson et al., 1995), and it enhanced xylulose growth rate in a strain with XYL1, XYL2 and XKS1 chromosomally integrated (Johansson and Hahn-Hägerdal, 2002). Enhanced expression level of TAL1 was also found in an arabinose-utilizing mutant of S. cerevisiae. (Becker and Boles/2003). In the present study, genes in both the oxidative and the non-oxidative pentose phosphate pathway were up-regulated in C1, C5 and BH42. In addition, several non-oxidative PPP genes were indigenously highly expressed in TMB3399, which might explain why they were not further enhanced in TMB3400. Up-regulated pentose phosphate pathway gene expression was observed also during glucose growth (data not shown), indicating that the changed gene expression reflects the capability of these strains to grow on xylose.

### Galactose and Mating Metabolism.

In all xylose-growing strains up-regulated galactose metabolism was associated with down-regulated mating metabolism. Altered mating metabolism might be a secondary effect of modified galactose metabolism. For example, a GAL4 over-expressing strain showed a decreased expression level of MFα1, involved in mating (Bro et al., 2004). Similarly GAL11, which is a component of the RNA polymerase II holoenzyme and a positive and negative transcriptional regulator of genes in mating-type specialization, was up-regulated in C1, C5 and TMB3400.

When a deletion was made in the GAL11 locus, it resulted in defects in mating (Nishizawa et al., 1990).

### CONCLUSIONS

Changes have occurred in various parts of the metabolism in the xylose growing S. cerevisiae strains, suggesting that several simultaneous modifications are required to optimize the strain for xylose growth. These modifications should notably include sufficient transport capacity, sufficient flux though the oxidative and the non-oxidative pentose phosphate pathway and efficient steps for NADPH and NAD⁺ regeneration. The up-regulation of the whole galactose pathway and the down-regulation of genes in the mating cascade are most probably not directly involved in growth on xylose.

### REFERENCES

Affymetrix Gene Expression Monitoring, GeneChip Expression Analysis, Technical Manual, Available on www.Affymetrix.com
Affymetrix (2002). Statistical Algorithms Description Document. Available on www.Affymetrix.com.
Affymetrix (2003). GeneChip Expression Analysis, Data Analysis Fundamentals: Available on www.affymetrix.com.
Alberti, A., T. Lodi, I. Ferrero and C. Donnini (2003). MIG1-dependent and MIG1-independent regulation of GAL gene expression in Saccharomyces cerevisiae: role of Imp2p. Yeast 20(13): 1085-1096.
Becker, J. and E. Boles (2003). A modified Saccharomyces cerevisiae strain that consumes L-Arabinose and produces ethanol. Appl Environ Microbiol 69(7): 4144-4150.
Bro, C., S. Knudsen, B. Regenberg, L. Olsson and 3. Nielsen (2004). Identification of novel metabolic engineering targets by using genome-wide transcription analysis. Submitted.
Bruinenberg, P., P. de Bot, J. van Dijken and A. Scheffers (1983). The role of redox balances in the anaerobic fermentation of xylose by yeasts. Eur J Appl Microbiol Biotechnol 18: 287-292.
Busturia, A. and R. Lagunas (1986). Catabolite inactivation of the glucose transport system in Saccharomyces cerevisiae. J Gen Microbiol 132 (Pt 2): 379-385.
Cronwright, G. (2002). Personal Communication.
Donnini, C., T. Lodi, I. Ferrero and P. P. Puglisi (1992). IMP2, a nuclear gene controlling the mitochondrial dependence of galactose, maltose and raffinose utilization in Saccharomyces cerevisiae. Yeast 8(2): 83-93.
Eliasson, A., E. Boles, B. Johansson, M. Österberg, J. M. Thevelein, I. Spencer-Martins, H. Juhnke and B. Hahn-Hägerdal (2000a). Xylulose fermentation by mutant and wild-type strains of Zygosaccharomyces and Saccharomyces cerevisiae. Appl Microbiol Biotechnol 53(4): 376-382.
Eliasson, A., C. Christensson, C. F. Wahlbom and B. Hahn-Hägerdal (2000b). Anaerobic xylose fermentation by recombinant Saccharomyces cerevisiae carrying XYL1, XYL2, and XKS1 in mineral medium chemostat cultures. Appl Environ Microbiol 66(8): 3381-3386.
Elion, E. A. (2000). Pheromone response, mating and cell biology. Curr Opin Microbiol 3(6): 573-581.
Erasmus, D. J., G. K. van der Merwe and H. J. J. Van Vuuren (2003). Genome-wide expression analyses: Metabolic adaptation of Saccharomyces cerevisiae to high sugar stress. FEMS Yeast Res 3(4): 375-399.
Gárdonyi, M., M. Jeppsson, G. Lidén, M. F. Gorwa-Grauslund and B. Hahn-Hägerdal (2003). Control of xylose consumption by xylose transport in recombinant Saccharomyces cerevisiae. Biotechnol Bioeng 82(7): 818-824.
Goffrini, P., I. Ferrero and C. Donnini (2002). Respiration-dependent utilization of sugars in yeasts: a determinant role for sugar transporters. J Bacteriol 184(2): 427-32.
Hahn-Hägerdal, B., C. F. Wahlbom, M. Gárdonyi, W. H. van Zyl, R. R. Cordero Otero and L. J. Jönsson (2001). Metabolic engineering of Saccharomyces cerevisiae for xylose utilization. Adv Biochem Eng Biotechnol 73: 53-84.
Hamacher, T., J. Becker, M. Gárdonyl, B. Hahn-Hägerdal and E. Boles (2002). Characterization of the xylose-transporting properties of yeast hexose transporters and their influence on xylose utilization. Microbiology 148(Pt 9): 2783-2788.
Horak, J. and D. H. Wolf (1997). Catabolite inactivation of the galactose transporter in the yeast Saccharomyces cerevisiae: ubiquitination, endocytosis, and degradation in the vacuole. J Bacteriol 179(5): 1541-1549.
Horak, J. and D. H. Wolf (2001). Glucose-induced monoubiquitination of the Saccharomyces cerevisiae galactose transporter is sufficient to signal its internalization. J Bacteriol 183(10): 3083-3088.
Horak, J., J. Regelmann and D. H. Wolf (2002). Two distinct proteolytic systems responsible for glucose-induced degradation of fructose-1,6-bisphosphatase and the Gal2p transporter in the yeast Saccharomyces cerevisiae share the same protein components of the glucose signaling pathway._J Biol Chem 277(10): 8248-8254.
Jeppsson, M., B. Johansson, B. Hahn-Hägerdal and M. F. Gorwa-Grauslund (2002). Reduced oxidative pentose phosphate pathway flux in recombinant xylose-utilizing Saccharomyces cerevisiae strains improves the ethanol yield from xylose. Appl Environ Microbiol 68(4): 1604-1609.
Jeppsson, M., B. Johansson, P. Ruhdal-Jensen, B. Hahn-Hägerdal and M. F. Gorwa-Grauslund (2003a). The level of glucose-6-phosphate dehydrogenase activity strongly influences xylose fermentation and inhibitor sensitivity in recombinant Saccharomyces cerevisiae strains. YEAST 20: 1263-1272.
Jeppsson, M., K. Träff, B. Johansson, B. Hahn-Hägerdal and M. F. Gorwa-Grauslund (2003b). Effect of enhanced xylose reductase activity on xylose consumption and product distribution in xylose-fermenting recombinant Saccharomyces cerevisiae. FEMS Yeast Res 3: 167-175.
Johansson, B., C. Christensson, T. Hobley and B. Hahn-Hägerdal (2001). Xylulokinase overexpression in two strains of Saccharomyces cerevisiae also expressing xylose reductase and xylitol dehydrogenase and its effect on fermentation of xylose and lignocellulosic hydrolysate. Appl Environ Microbiol 67(9): 4249-4255.
Johansson, B. and B. Hahn-Hägerdal (2002). The non-oxidative pentose phosphate pathway controls the fermentation rate of xylulose but not of xylose in Saccharomyces cerevisiae TMB3001. FEMS Yeast Res 2(3): 277-282.
Johnston, M. (1987). A model fungal gene regulatory mechanism: the GAL genes of Saccharomyces cerevisiae. Microbiol. Rev 51(4): 458-476.
Kawai, S., S. Suzuki, S. Mori and K. Murata (2001). Molecular cloning and identification of UTR1 of a yeast Saccharomyces cerevisiae as a gene encoding an NAD kinase. FEMS Microbiol Lett 200(2): 181-184.
Kuhn, A., C. van Zyl, A. van Tonder and B. A. Prior (1995). Purification and partial characterization of an aldo-keto reductase from Saccharomyces cerevisiae. Appl Environ Microbiol 61(4): 1580-1585.
Kötter, P. and M. Ciriacy (1993). Xylose fermentation by Saccharomyces cerevisiae. Appl Microbiol Biotechnol 38: 776-783.
Lee, T.-H., M.-D. Kim, Y.-C. Park, S.-M. Bae, Y.-W. Ryu and J.-H. Seo (2003). Effects of xylulokinase activity on ethanol production from D-xylulose by recombinant Saccharomyces cerevisiae. 3 Appl Microbiol 95(4): 847-852.
Lohr, D., P. Venkov and J. Zlatanova (1995). Transcriptional regulation in the yeast GAL gene family: a complex genetic network. Faseb J 9(9): 777-787.
Nehlin, J. O., M. Carlberg and H. Ronne (1991). Control of yeast GAL genes by MIG1 repressor: a transcriptional cascade in the glucose response. Embo J 10(11): 3373-3377.
Nishizawa, M., Y. Suzuki, Y. Nogi, K. Matsumoto and T. Fukasawa (1990). Yeast Gal11 protein mediates the transcriptional activation signal of two different transacting factors, Gal4 and general regulatory factor I/repressor/activator site binding protein 1/translation upstream factor. Proc Natl Acad Sci USA 87(14): 5373-5377.
Piper, M. D., P. Daran-Lapujade, C. Bro, B. Regenberg, S. Knudsen, J. Nielsen and J. T. Pronk (2002). Reproducibility of oligonucleotide microarray transcriptome analyses. An interlaboratory comparison using chemostat cultures of Saccharomyces cerevisiae. J Biol Chem 277(40): 37001-37008.
Ren, B., F. Robert, J. J. Wyrick, O. Aparicio, E. G. Jennings, I. Simon, J. Zeitlinger, J. Schreiber, N. Hannett, E. Kanin, T. L. Volkert, C. J. Wilson, S. P. Bell and R. A. Young (2000). Genome-wide location and function of DNA binding proteins. Science 290(5500): 2306-2309.
Schmitt, M. E., T. A. Brown and B. L. Trumpower (1990). A rapid and simple method for preparation of RNA from Saccharomyces cerevisiae. Nucleic Acids Res 18(10): 3091-3092.
Sedlak, M., H. J. Edenberg and N. W. Y. Ho (2003). DNA microarray analysis of the expression of the genes encoding the major enzymes in ethanol production during glucose and xylose co-fermentation by metabolically engineered Saccharomyces yeast. Enzyme Microb. Technol. 33(1): 19-28.
Sonderegger, M. and U. Sauer (2003). Evolutionary engineering of Saccharomyces cerevisiae for anaerobic growth on xylose. Appl Environ Microbiol 69(4): 1990-1998.
Sonderegger, M., M. Jeppsson, B. Hahn-Hägerdal and U. Sauer (2004a). The molecular basis for anaerobic growth of Saccharomyces cerevisiae on xylose investigated by global gene expression and metabolic flux analysis. Accepted for publication in Appl Environ Microbiol.
Sonderegger, M., M. Jeppsson, C. Larsson, M. F. Gorwa-Grauslund, E. Boles, L. Olsson, I. Spencer-Martins, B. Hahn-Hägerdal and U. Sauer (2004b). Fermentation performance of engineered and evolved xylose-fermenting Saccharomyces cerevisiae strains. Accepted for publication in Biotechnol Bioeng.
Spencer-Martins, I. (2003). Personal communication.
Stadler, J. A. and R. J. Schweyen (2002). The yeast iron regulon is induced upon cobalt stress and crucial for cobalt tolerance. J Biol Chem 277(42): 39649-39654.
ter Linde, J. J. M., H. Liang, R. W. Davis, H. Y. Steensma, J. P. van Dijken and J. T. Pronk (1999). Genome-wide transcriptional analysis of aerobic and anaerobic chemostat cultures of Saccharomyces cerevisiae. J Bacteriol 181(24): 7409-7413.
Toivari, M. H., A. Aristidou, L. Ruohonen and M. Penttilä (2001). Conversion of xylose to ethanol by recombinant Saccharomyces cerevisiae: Importance of xylulokinase (XKS1) and oxygen Availability. Metab Eng 3(3): 236-249.
Träff, K. L., L. J. Jönsson and B. Hahn-Hägerdal (2002). Putative xylose and arabinose reductases in Saccharomyces cerevisiae. Yeast 19(14): 1233-1241.
Wahlbom, C. F., A. Eliasson and B. Hahn-Hägerdal (2001). Intracellular fluxes in a recombinant xylose-utilizing Saccharomyces cerevisiae cultivated anaerobically at different dilution rates and feed concentrations. Biotechnol Bioeng 72(3): 289-296.
Wahlbom, C. F., W. H. van Zyl, L. J. Jönsson, B. Hahn-Hagerdal and R. R. Cordero Otero (2003a). Generation of the improved recombinant xylose-utilizing Saccharomyces cerevisiae TMB 3400 by random mutagenesis and physiological comparison with Pichia stipitis CBS 6054, FEMS Yeast Res 3(3): 319-326.
Wahlbom, C. F., R. R. Cordero Otero, W. H. Van Zyl, B. Hahn-Hägerdal and L. J. Jönsson (2003b). Molecular analysis of a Saccharomyces cerevisiae mutant with improved ability to utilize xylose shows enhanced expression of proteins involved in transport, initial xylose metabolism, and the pentose phosphate pathway. Appl Environ Microbiol 69(2): 740-746.
Walfridsson, M., J. Hallborn, M. Penttilä, S. Keränen and B. Hahn-Hägerdal (1995). Xylose-metabolizing Saccharomyces cerevisiae strains overexpressing the TKL1 and TAL1 genes encoding the pentose phosphate pathway enzymes transketolase and transaldolase. Appl Environ Microbiol 61(12): 4184-4190.
Verduyn, C., E. Postma, W. A. Scheffers and J. P. van Dijken (1992). Effect of benzoic acid on metabolic fluxes in yeasts: a continuous-culture study on the regulation of respiration and alcoholic fermentation. Yeast 8(7): 501-517.

1. A new xylose-utilizing Saccharomyces cerevisiae strain by expression of xylose reductase, xylose dehydrogenase, and xylose kinase (XR-XDH-XK) or xylose isomerase (XI) genes fermenting xylose to ethanol better than a control strain having
a) increased transporting capacity with regard to galactose,
b) increased transporting capacity with regard to xylose,
c) increased conversion capacity of xylulose to xylulose-5P
d) increased activity of the oxidative pentose phosphate pathway, and/or
e) increased activity of the non-oxidative pentose phosphate pathway, whereby
i) the strain is up-regulated with regard to the gene YEL041W and/or the gene YLR152C to provide an increased level of NAD(H)⁺ kinase,
ii) the strain is up-regulated with regard to genes GAL1, Gal7, and GAL10 to provide for galactose metabolism,
iii) the strain up-regulated with regard to the gene GAL2 to provide for galactose transport
iv) the strain is up-regulated with regard to the genes XYL1, XYL2, and XKS1 when the strain is a chromosomally integrated strain to provide for increased activity of the oxidative pentose phosphate pathway.

2. A new S. cerevisiae strain according to claim 1, wherein the genes SOL1, SOL2, SOL3, SOL4, ZWF1 and/or GND1 are up regulated to provide for an increased level of glucose-6-phosphatase dehydrogenase, and phosphogluconate dehyrogenase.

3. A new S. cerevisiae strain according to claim 1, wherein the gene TAL1 is upregulated to provide for an increased level of transaldolase, the gene TKL1 to provide for an increased level of transkelotase, the gene RPE1 to provide for an increased level of D-ribulose-5-phosphate-3-epimerase, and/or the gene RKI1 to provide for an increased level of D-ribose-5-phosphate-ketol-isomerase.

4. A new S. cerevisiae strain according to claim 1, wherein the gene PUT4 is upregulated.

5. A new S. cerevisiae strain according to claim 1, wherein the gene YOR202W is up-regulated.

6. A new S. cerevisiae strain according to claim 1, wherein two or more properties from claims 2-5 are combined.

7-11. (canceled)
